(19) United States
(12) Patent Application Publication
(10) Pub. No.: US 2006/0134610 A1
(43) Pub. Date: Jun. 22, 2006

(54) Chris-like virus, compositions, vaccines and uses thereof

(76) Inventors: Peter M. Palese, Leonia, NJ (US); Adolfo Garcia-Sastre, New York, NY (US); Christopher F. Basler, Bronx, NY (US)

Correspondence Address: CHERYL H AGRIS PHD, PO BOX 806, PELHAM, NY, 10803, US

(21) Appl. No.: 10/828,564

(22) Filed: Apr. 21, 2004

Related U.S. Application Data
(60) Provisional application No. 60/483,337, filed on Jun. 27, 2003.

(51) Int. Cl. C12Q 1/70; G01N 33/53; C07H 21/04; C12N 7/00; C07K 14/115

(52) U.S. Cl. 435/5; 435/69.3; 435/235.1; 435/325; 530/350; 530/388.3; 536/23.72

(57) ABSTRACT

A novel virus, the Chris-like virus, has been identified and characterised. Sequences derived from a new paramyxovirus, Chris-like virus (CV), are provided. These sequences encode viral proteins with similarity to recognized viral proteins in other known and characterized paramyxoviruses. The CV viral sequences and the polypeptides encoded therein are useful for the detection, immunological protection and therapy of CV. The sequences provided by the invention are also useful for the isolation and characterization of CV, for the propagation of CV or its viral components in cells or tissue culture, for the generation of immunogenic polypeptides and vaccines, and for the screening and identification of antiviral agents for CV.

### PRIORITY CLAIM

This application claims priority under 35 U.S.C. § 19(e) to provisional application Ser. No. 60/483,337, Filed Jun. 27, 2003, the contents of which are incorporated herein by reference.

### FIELD OF THE INVENTION

The present invention relates to the discovery of nucleic acid sequences encoding novel virus proteins of a new paramyxovirus, denoted Chris-like virus (CV). Specifically, the invention pertains to nucleic acid sequences encoding viral polypeptides of the Chris-like virus, amino acid sequences thereof, and uses thereof. The invention relates to diagnostic nucleic acid fragments, diagnostic proteins, diagnostic antibodies for the Chris-like virus and related viruses, and to the screening of e.g. antiviral agents. The invention also relates to protective polypeptides and vaccines that provide protection or elicit protective antibodies to CV infection, and to antibodies immunologically specific for these polypeptides for use in therapy. Furthermore, the invention relates to antiviral agents that modulate replication of said virus.

### BACKGROUND OF THE INVENTION

Paramyxoviridae is a virus family containing enveloped single-stranded RNA of the negative-sense genome and includes the viruses of Newcastle disease virus (NDV), parainfluenza virus, Sendai virus, simian virus 5, Nipah virus, measles virus and mumps virus. The family of Paramyxoviridae contains viruses that induce a wide range of distinct clinical illnesses in humans. These include measles virus, which in rare instances is followed by subacute sclerosing panencephalitis (SSPE); mumps virus, which induces symptoms of parotitis, orchitis and encephalitis, and the parainfluenza viruses which are respiratory pathogens.

Members of the Paramyxoviridae family are enveloped viruses containing a linear, single-strand, nonsegmented, negative sense RNA genome. The virus envelope is a lipid bilayer, studded with virus encoded glycoproteins which have properties of haemagglutination and fusion (the F protein). The genomic RNA contains genes for NP, P, M, F, an attachment protein such as HN, H or G, and L. The genomic RNA also contains a leader sequence at the 3′ end. Sequences at the end of the genome are involved in transcription and replication of the RNA by the viral RNA-dependent RNA polymerase. In addition, intergenic junctions contain gene-end, polyadenylation and gene-start signals.

The structural elements of the virion include the virus envelope that is a lipid bilayer derived from the cell plasma membrane. The glycoprotein, hemagglutinin-neuraminidase (HN), protrudes from the envelope allowing the virus to contain both hemagglutinin and neuraminidase activities; in some paramyxoviruses this attachment protein lacks neuraminidase activity. The fusion glycoprotein (F), which also interacts with the viral membrane, is first produced as an inactive precursor, then cleaved post-translationally to produce two disulfide linked polypeptides. The active F protein is involved in penetration of virus into host cells by facilitating fusion of the viral envelope with the host cell plasma membrane and represents an attractive vaccine or immunogenic polypeptide candidate. The matrix protein (M), is involved with viral assembly, and interacts with both the viral membrane as well as the nucleocapsid proteins.

The main protein subunit of the nucleocapsid is the nucleocapsid protein (NP) which confers helical symmetry on the capsid. In association with the nucleocapsid are the P and L proteins. The phosphoprotein (P), which is subject to phosphorylation, is thought to play a regulatory role in transcription. The L gene, which encodes an RNA-dependent RNA polymerase, is required for viral RNA synthesis together with the P protein. The L protein, which takes up nearly half of the coding capacity of the viral genome is the largest of the viral proteins, and plays an important role in both transcription and replication. The P sequences of the virus can also encode additional viral proteins, the V and W proteins, which are generated by RNA editing through the addition of one or two G residues, resulting in a shift to the reading frame. In certain paramyxoviruses, the genomic RNA encodes a V protein and the P protein is generated through RNA editing. In addition, the P sequences can encode a C protein through a second reading frame.

The replication of all negative-strand RNA viruses is complicated by the absence of cellular machinery required to replicate RNA. Additionally, the negative-strand genome cannot be translated directly into protein, but must first be transcribed into a positive-strand (mRNA) copy. Therefore, upon entry into a host cell, the genomic RNA alone cannot synthesize the required RNA-dependent RNA polymerase. The L, P and NP proteins must enter the cell along with the genome on infection.

It is hypothesized that most or all of the viral proteins that transcribe Paramyxoviridae mRNA also carry out their replication. Directly following penetration of the virus, transcription is initiated by the L protein using the negative-sense RNA in the nucleocapsid as a template. Viral RNA synthesis is regulated such that it produces monocistronic mRNAs during transcription.

Following transcription, virus genome replication is the second essential event in infection by negative-strand RNA viruses. Virus genome replication is mediated by virus-specified proteins. The first products of replicative RNA synthesis are complementary copies (i.e., plus-polarity) of virus genome RNA (cRNA). These plus-stranded copies (anti-genomes) differ from the plus-strand mRNA transcripts in the structure of their termini. Unlike the mRNA transcripts, the anti-genomic cRNAs are not capped and methylated at the 5′ termini, and are not truncated and polyadenylated at the 3′ termini. The cRNAs are coterminal with their negative strand templates and contain all the genetic information in each genomic RNA segment in the complementary form. The cRNAs serve as templates for the synthesis of negative-strand viral genomes (vRNAs).

Reverse genetics systems which allow the genetic manipulation of the paramyxovirus NDV genome from recombinant or cloned DNA have been described (Peeters B P et al. (1999) J Virol 73: 5001-5009; Romer-Oberdorfer A et al. (1999) J. Gen. Virol. 80: 2987-2995; Krishnamurthy S et al. (2000) Virology 278: 168-182; Nakaya T. et al. (2001) J. Virol. 75: 11868-11873, U.S. Pat. Pub. No. 2003/0224077). Recombinant systems for other non-segmented negative strand RNA viruses have also been described, including rabies virus (Schnell M J et al. (1994) EMBO J. 13: 4195-4203), VSV (Lawson N D et al. (1995) Proc. Natl. Acad. Sci. USA 92: 4477-4481; Whelan S P et al. (1995) Proc. Natl. Acad. Sci. (USA) 92: 8388-8392), measles virus (Radecke F et al. (1995) EMBO J 14: 5773-5784), infectious human respiratory syncytial virus (Collins P L et al. (1995) Proc. Natl. Acad. Sci. (USA) 92: 11563-11567), Sendai virus (Garcin D et al. (1995) EMBO J 14: 6087-6094; Kato A et al. (1996) Genes Cells 1: 569-579), rinderpest virus (Baron M D and Barrett T. (1997) J Virol 71: 1265-1271), parainfluenza virus (Hoffman M A, and Banerjee A K. (1997) J Virol 71: 4272-4277; Durbin A P et al. (1997) Virology 235: 323-332) and paramyxovirus SV5 (He B et al. (1997) Virology 1997; 237: 249-260). In all these systems, the necessary viral proteins of the non-segmented genome (NP, P/V, M, F, HN and L) are encoded from a single plasmid or recombinant nucleic acid on a single RNA molecule, thus mimicking the viral genome organization. In addition, the viral NP, P and L proteins were expressed from cotransfected plasmids or their function provided by co-expressed heterologous RNA polymerase or by helper virus.

It has recently been found that paramyxoviruses have oncolytic properties (U.S. Pat. Pub. No. 2003/004384). Specifically, NDV has been found to cause regression of human cancer cells derived from sarcomas, melanomas, breast carcinomas, ovarian carcinomas, bladder carcinomas, colon carcinoma, prostate carcinoma, small cell and non-small cell lung carcinomas, and glioblastomas, neuroblastoma and fibrosarcoma (U.S. Patent Publication 2003/0044384, Sinkovics et al., 2000, J. Clin. Virol. 16:1-15 and Phuangsab et al., 2001, Cancer Lett. 172:27-36) and measles virus has been found to be potent against ovarian cancer cells and myeloma cells (Peng et al., 2002, Cancer Res. 15:4656-62 and Peng et al., 2003, Blood, 101:2557-62).

Vaccines

Vaccination has proven to be a successful means for conferring immune protection against many human pathogens. In the search for safe and effective vaccines for immunizing individuals against infective pathogenic agents such as viruses, bacteria, and infective eukaryotic organisms, several strategies have been employed thus far. Each strategy aims to achieve the goal of protecting the individual against pathogen infection by administering to the individual a target protein that is associated with the pathogen and can elicit an immune response. Thus, when the individual is challenged by the infective pathogen, the individual's immune system recognizes the protein and mounts an effective defense against infection by the pathogen. There are several vaccine strategies for presenting pathogen proteins which include presenting the protein as part of a non-infective or less infective (attenuated) agent or as a discreet protein composition. Attenuation can be achieved by selection of a mutant virus or generation of altered virus through recombinant means. A particularly attractive target for attenuation of the paramyxoviruses is the V protein, which has anti-interferon activity. Altering this sequence by mutation results in an attenuated virus by virtue of its inability to block the host's interferon response. This also provides a target for drug interference or intervention to reduce the infectivity and severity of host infection.

Another means of immunizing against pathogens is provided by recombinant vaccines. One type of recombinant vaccine is attenuated by design (specific genes are mutated or deleted for instance) and requires the administration of an active, non-virulent infective agent which, upon establishing itself in a host, produces or causes to be produced antigens used to elicit the immune response. An additional type of recombinant vaccine employs non-virulent vectors that carry genetic material that encodes target antigens. This type recombinant vaccine similarly requires the administration of an active infective non-virulent agent which, upon establishing itself in a host, produces or causes to be produced, the antigen used to elicit the immune response. Such vaccines essentially employ non-virulent agents to present pathogen antigens that can then serve as targets for an anti-pathogen immune response. For example, the development of vaccinia as an expression system for vaccination has theoretically simplified the safety and development of infectious vaccination strategies with broader T-cell immune responses.

Another method of immunizing against infection uses subunit vaccines. Subunit vaccines generally consist of one or more isolated proteins derived from the pathogen. These proteins act as target antigens against which an immune response may be mounted by an individual. The proteins selected for subunit vaccine are displayed by the pathogen so that upon infection of an individual by the pathogen, the individual's immune system recognizes the pathogen and mounts a defense against it. Because subunit vaccines are not whole infective agents, they are incapable of becoming infective. Thus, they present no risk of undesirable virulent infectivity that is associated with other types of vaccines.

The citation of references herein shall not be construed as an admission that such is prior art to the present invention.

### SUMMARY OF THE INVENTION

The invention pertains to the identification and characterization of a newly discovered paramyxovirus, Chris-like virus (CV), its nucleotide sequences, its protein sequences and resulting polynucleotides and polypeptides. Studies of the sequence information contained within the CV sequences indicate that CV is a negative-stranded RNA virus that appears to be a new member of the Paramyxoviridiae family of viruses.

CV sequences and portions thereof are useful as probes to diagnose the presence of virus in samples, and to isolate naturally occurring variants of the virus. These sequences also make available polypeptide sequences of CV antigens encoded within the CV genome and permit the production of polypeptides that are useful as standards or reagents in diagnostic tests and/or as components of vaccines. Antibodies, including for example both polyclonal and monoclonal, directed against CV epitopes contained within these polypeptide sequences are also useful for diagnostic tests, as therapeutic agents, for screening of antiviral agents, and for the isolation of the CV agent from which these sequences derive. In addition, by utilizing probes derived from these sequences it is possible to isolate and sequence other portions of the CV genome, thus giving rise to additional probes and polypeptides which are useful in the diagnosis and/or treatment, both prophylactic and therapeutic, of CV and of related viruses.

Accordingly with respect to polynucleotides, some aspects of the invention are: an isolated CV nucleic acid molecule, a nucleic acid construct, an expression vector or host cell comprising a sequence derived from a CV genome or from CV RNA; a recombinant polynucleotide encoding an epitope of CV; a recombinant vector containing any of the above recombinant polynucleotides, and a host cell transformed with any of these vectors.

The present invention also relates to an isolated nucleic acid molecule comprising (a) a nucleic acid sequence hybridizing under stringent conditions to a nucleic acid sequence SEQ ID NOS: 3, 7 and 8; (b) a nucleic acid sequence having at least least 90% identity to an amino acid sequence comprising SEQ ID NOS: 3, 7 and 8; (c) a nucleic acid sequence encoding a polypeptide having at least about 90% identity to a polypeptide comprising SEQ ID NOS: 9, 10, 11, 12, 13 or 14.

Still other aspects of the invention are: isolated CV, a preparation of polypeptides from the isolated CV; an isolated CV polypeptide; an isolated polypeptide comprising an epitope which is immunologically identifiable with an epitope contained in CV.

Included aspects of the invention are a recombinant CV polypeptide; a recombinant polypeptide comprised of a CV epitope; and a fusion polypeptide comprised of a CV polypeptide. In particular aspects, polypeptides comprise all or a portion of the CV proteins selected from the group of M, P, F, V, C and W comprising amino acid sequences depicted in SEQ ID NOS: 9, 10, 11, 12, 13, 14, respectively.

The present invention likewise extends to the development of antibodies that bind to at least one epitope on a Chris-like virus and may be against the CV protein(s), or antigenic or immunogenic fragments thereof, including naturally raised and recombinantly prepared antibodies. Such antibodies could include both polyclonal and monoclonal antibodies prepared by known techniques, as well as bi-specific (chimeric) antibodies, anti-idiotype antibodies comprising a region which mimics a CV epitope and antibodies including other functionalities suiting them for additional diagnostic use conjunctive with their capability of modulating CV protein activity.

Yet another aspect of the invention is a method for producing antibodies to CV comprising administering to an individual an isolated immunogenic polypeptide containing a CV epitope or nucleic acid molecule encoding said polypeptide in an amount sufficient to produce an immune response.

Other aspects of the invention are: a polypeptide comprising a CV epitope, attached to a solid substrate; and an antibody to a CV epitope, attached to a solid substrate. These may be incorporated into kits.

Still other aspects of the invention include a method for producing a CV polypeptide comprising incubating host cells comprising a nucleic acid sequence encoding said polypeptide under conditions which allow expression of said polypeptide and isolating said polypeptide.

The invention also includes a methods and kits for detecting CV nucleic acids in a sample comprising contacting a nucleic acid molecule from a sample from a subject with at least one CV primer or probe and detecting the presence or absence of a paramyxovirus nucleic acid molecule in said sample to detect the presence or absence of said paramyxovirus. In a specific embodiment, a sample is contacted with a CV probe derived from the under conditions in which the polynucleotide will selectively hybridize to said paramyxovirus nucleic acid sequence and detecting hybridization of the nucleic acid molecule with said probe in said sample, wherein the detection of the hybridization indicates the presence of paramyxovirus in the sample. In another embodiment, the sample is contacted with at least one CV primer to provide an amplification mixture wherein said primer is sufficient in length to selectively hybridize to said paramyxovirus nucleic acid sequence and wherein said primer is capable of amplifying a detectable part of a paramyxovirus nucleic acid if said paramyxovirus is present in the sample; subjecting the amplification mixture to nucleic acid amplification and detecting whether a part of a paramyxovirus nucleic acid molecule has been amplified wherein detection of a part indicates the presence of paramyxovirus in the sample.

Immunoassays are also included in the invention. These include an immunoassay for detecting a CV antigen comprising incubating a sample suspected of containing a CV antigen with a probe antibody directed against the CV antigen to be detected under conditions which allow the formation of an antigen-antibody complex; and detecting an antigen-antibody complex containing the probe antibody. An immunoassay for detecting antibodies directed against a CV antigen comprises incubating a sample suspected of containing anti-CV antibodies with a probe polypeptide which contains an epitope of the CV, under conditions which allow the formation of an antibody-antigen complex; and detecting the antibody-antigen complex containing the probe antigen.

Also included in the invention are compositions for treatment or prevention of CV infection. In one embodiment, the composition comprises a polypeptide, an immunogenic peptide containing a CV epitope, or an inactivated preparation of CV, or an attenuated preparation of CV. Another aspect of the invention is a tissue culture grown cell infected with CV. The present invention also includes antiviral agents, specifically agents that modulate the replication of paramyxovirus, particularly, CV virus and/or production or expression of the CV nucleic acid molecule of the present invention as well as compositions comprising said agents. Such agents include but are not limited to peptide or peptide analogs that bind to the CV F protein, antisense oligonucleotides, ribozymes and siRNAs. The invention is further directed to the use of said polypeptide, nucleic acid molecule, CV virus, antiviral agent for the manufacture of a medicament for the treatment, prevention or amelioration of a medical condition.

The present invention also includes isolated Chris-like virus proteins having the activities noted herein, and that display the amino acid sequences set forth and described above and selected from the group consisting of M, P, F, V, C and W comprising amino acid sequences depicted in SEQ ID NOS. 9, 10, 11, 12, 13, 14, respectively. Analogs, mutants and variants, including naturally occurring variants of the CV proteins, including M, P, F, V, C and W are also included in the invention.

The invention includes an assay system for screening of potential drugs effective to modulate Chris-like virus protein activity, including in infected host cells, by interrupting or inhibiting the protein activity or expression. In one instance, the test drug could be administered to a cellular sample with the Chris-like virus protein or the virus itself or a viral expression system, to determine its effect upon the activity of the protein, the expression of the protein or the infectivity and replication of the virus, by comparison with a control. In yet a further embodiment, the invention contemplates antagonists of the activity of a Chris-like virus protein.

Furthermore, the invention includes a method for modulating tumor growth in a subject in need thereof comprising administering to said subject the CV virus of the present invention in an amount effective to modulate said tumor growth. In one embodiment, the CV virus is an attenuated virus as well as uses of said CV virus for the manufacture of a medicament for prevention, treatment or amelioration of a medical condition. Compositions comprising the CV of the present invention may also be administered to the subject.

Recombinant CV viruses comprising a heterologous nucleic acid molecule may also be obtained. The invention is directed to said viruses as well, host cells comprising said viruses and methods for obtaining said viruses. The recombinant viruses may be obtained by obtaining CV cDNA and inserting said heterologous sequence between CV genes or alternatively obtaining a nucleic acid sequence encoding a fusion protein of a CV protein and heterologous sequence.

The invention is further directed to host cells comprising said recombinant viruses, as cell as a method for obtaining a heterologous polypeptide comprising expressing said host cell and isolating said polypeptide.

Other objects and advantages will become apparent to those skilled in the art from a review of the following description that proceeds with reference to the following illustrative drawings.

### BRIEF DESCRIPTION OF THE DRAWINGS

FIG. 1 depicts a phylogenetic comparison of select F proteins from AY040225 and from Nipah virus, Hendra virus, Tupaia paramyxovirus and measles virus, generated by the Clustal Method using the DNAStar Megalign computer program.

FIG. 2 depicts the predicted Chris-like virus V protein sequence (the carboxy terminal region) (SEQ ID NO: 12). The Query sequence is from the translation of the modified Angrem104 in which a non-template encoded G has been added at the editing site. The Subject sequence is from the Nipah virus V protein (SEQ ID NO: 18). Bold characters highlight cysteines conserved between Chris-like virus and Nipah virus V proteins. Underlined residues are conserved among many paramyxovirus V proteins.

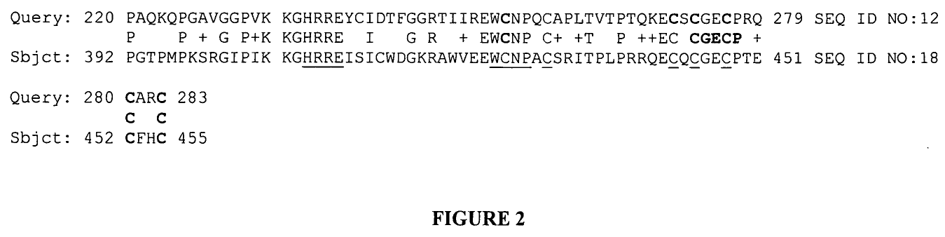

FIG. 3 provides a phylogenetic comparison of select V proteins from AF367870 and from Nipah virus, Hendra virus, Tupaia paramyxovirus and measles virus, generated by the Clustal Method using the DNAStar Megalign computer program.

FIG. 4 provides a phylogenetic comparison of the amino-terminal 210 amino acids from select V proteins from AF367870 and from Nipah virus, Hendra virus, Tupaia paramyxovirus and measles virus, generated by the Clustal Method using the DNAStar Megalign computer program.

FIG. 5 provides a phylogenetic comparison of select M proteins from AY040225 and from Nipah virus, Hendra virus, Tupaia paramyxovirus and measles virus, generated by the Clustal Method using the DNAStar Megalign computer program.

FIG. 6 depicts the Chris-like virus fusion protein fusion peptide (SEQ ID NO:11) and heptad repeats. The putative fusion peptide of Chris-like virus fusion protein is in bold, the putative heptad repeat A is in ITALICS and the putative heptad repeat B is in bold and underlined.

### DEFINITIONS AND TERMS

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The terms “Chris-like virus”, “CV” and any variants not specifically listed may be used herein interchangeably, and as used throughout the present application and claims refer to the novel paramyxovirus described herein. The Chris-like virus as described and characterized herein includes and encompasses its single or multiple viral proteins, and extends to those proteins, particularly including any of the M, P, F, V, C and W comprising amino acid sequences having at least 90% identity to SEQ ID NOS: 9, 10, 11, 12, 13 or 14, respectively, having the amino acid sequence data described herein and the profile of activities set forth herein. Accordingly, proteins displaying substantially equivalent activity are likewise contemplated. Also, the terms “M”, “P”, “F”, “V”, “C” and “W” Chris-like virus proteins each are intended to include within their scope proteins specifically recited herein as well as all analogs having substantially identical sequences and allelic variants.

As defined herein, a “gene” is the segment of DNA involved in producing a polypeptide chain; it includes regions preceding and following the coding region, as well as intervening sequences (introns) between individual coding segments (exons).

As defined herein “isolated” refers to material removed from its original environment and is thus altered “by the hand of man” from its natural state.

As defined herein, “modulate” means to modify the rate and/or amount of e.g., replication of a virus.

The amino acid residues described herein are preferred to be in the “L” isomeric form. However, residues in the “D” isomeric form can be substituted for any L-amino acid residue, as long as the desired functional is retained by the polypeptide. NH₂ refers to the free amino group present at the amino terminus of a polypeptide. COOH refers to the free carboxy group present at the carboxy terminus of a polypeptide.

“Nucleic acid construct” is defined herein as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term expression cassette when the nucleic acid construct contains all the control sequences required for expression of a coding sequence of the present invention.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5′-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” region of a recombinant virus is an identifiable segment of nucleic acid within a larger nucleic acid molecule that is not found in association with the larger molecule in nature.

An “expression vector” may be any vector (e.g., a plasmid or virus) which can be conveniently subjected to recombinant DNA procedures and can bring about the expression of the nucleic acid sequence.

An “origin of replication” refers to those DNA sequences that participate in DNA synthesis.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

It should be appreciated that also within the scope of the present invention are DNA sequences encoding CV proteins, which code for a polypeptide having the same amino acid sequence as the sequences disclosed herein, but which are degenerate to the nucleic acids disclosed herein. By “degenerate to” is meant that a different three-letter codon is used to specify a particular amino acid.

Mutations can be made in the CV sequences disclosed herein such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes that do not significantly alter the activity or binding characteristics of the resulting protein. Particularly preferred substitutions are: Lys for Arg and vice versa such that a positive charge may be maintained; Glu for Asp and vice versa such that a negative charge may be maintained; Ser for Thr such that a free —OH can be maintained; and Gln for Asn such that a free NH₂ can be maintained. Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces ∃-turns in the protein's structure.

As used herein, “epitope” refers to an antigenic determinant of a polypeptide. An epitope could comprise 3 amino acids in a spatial conformation which is unique to the epitope. Generally an epitope consists of at least 5 such amino acids, and more usually, consists of at least 8-10 such amino acids. Methods of determining the spatial conformation of amino acids are known in the art, and include, for example, x-ray crystallography and 2-dimensional nuclear magnetic resonance.

A polypeptide is “immunologically reactive” with an antibody when it binds to an antibody due to antibody recognition of a specific epitope contained within the polypeptide. Immunological reactivity may be determined by antibody binding, more particularly by the kinetics of antibody binding, and/or by competition in binding using as competitor(s) a known polypeptide(s) containing an epitope against which the antibody is directed. The techniques for determining whether a polypeptide is immunologically reactive with an antibody are known in the art.

An “antibody” is any immunoglobulin, including antibodies and fragments thereof, that bind a specific epitope. The term encompasses polyclonal, monoclonal, and chimeric antibodies; the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

Fab and F(ab′)₂ fragments are prepared by the proteolytic reaction of papain and pepsin, respectively, on substantially intact antibody molecules by methods that are well-known. See for example, U.S. Pat. No. 4,342,566. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

Another example is “altered antibodies”, which refers to antibodies in which the naturally occurring amino acid sequence in a vertebrate antibody has been varied. Utilizing recombinant DNA techniques, antibodies can be redesigned to obtain desired characteristics. The possible variations are many, and range from the changing of one or more amino acids to the complete redesign of a region, for example, the constant region. Changes in the constant region, in general, to attain desired cellular process characteristics, e.g., changes in complement fixation, interaction with membranes, and other effector functions. Changes in the variable region may be made to alter antigen binding characteristics. The antibody may also be engineered to aid the specific delivery of a molecule or substance to a specific cell or tissue site. The desired alterations may be made by known techniques in molecular biology, e.g., recombinant techniques, site directed mutagenesis, etc.

As used herein, the term “immunogenic polypeptide” is a polypeptide that elicits a cellular and/or humoral immune response, whether alone or linked to a carrier in the presence or absence of an adjuvant.

The term “polypeptide” refers to a polymer of amino acids and does not refer to a specific length of the product; thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring.

A nucleic acid molecule is “operatively linked” to an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

The term “stringent hybridization conditions” are known to those skilled in the art and can be found in Current Protocols in Molecular Biology, John Wiley & Sons, N.Y. (1989), 6.3.1-6.3.6. A preferred, non-limiting example of stringent hybridization conditions is hybridization in 6× sodium chloride/sodium citrate (SSC) at about 45° C., followed by one or more washes in 0.2.×SSC, 0.1% SDS at 50° C., preferably at 55° C., and more preferably at 60° C. or 65° C.

As defined herein a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

### DETAILED DESCRIPTION OF THE INVENTION

In its primary aspect, the present invention concerns the identification of a novel paramyxovirus, the Chris-like virus (CV), as well as its nucleic acid and polypeptide sequences of CV. Said virus may be present in the tissue or organ of a subject as well as in bodily fluids or blood products. The isolated virus of the present invention contains a nucleic acid molecule: (a) hybridizing under stringent conditions to SEQ ID NOS: 3, 7 and/or 8 a reverse complement thereof; (b) having at least 90% identity with SEQ ID NOS: 3, 7 and/or 8 using a GAP algorithm with default parameters or a reverse complement thereof and/or (c) encoding a polypeptide having an amino acid sequence depicted in SEQ ID NOS: 9, 10, 11, 12, 13 and/or 14 or reverse complements thereof. Said virus may also contain a polypeptide having at least 90% identity with SEQ ID NOS: 9, 10, 11, 12, 13 or 14 using a GAP algorithm with default parameters.

In addition to the above, the information provided herein allows the identification of additional CV strains or isolates. The isolation and characterization of the additional CV strains or isolates may be accomplished by isolating the nucleic acids from body components which contain viral particles and/or viral RNA, creating libraries using polynucleotide probes based on the CV probes described herein, screening the libraries for clones containing CV sequences, and comparing the CV sequences from the new isolates with the sequences described herein. The polypeptides encoded therein, or in the viral genome, may be monitored for immunological cross-reactivity utilizing the polypeptides and antibodies described herein. Other methods for identifying CV strains will be obvious to those of skill in the art, based upon the information provided herein.

Nucleic Acid Molecules

As stated above, the present invention also relates to a nucleic acid molecule, particularly a recombinant DNA molecule or cloned gene, or a degenerate variant thereof, which encodes a CV viral protein, or a fragment thereof, that possesses an amino acid sequence as set forth herein, including the sequences or a portion thereof of the Chris-like virus M, P, F, V, C and W as described herein, particularly as embodied in SEQ ID NOS: 9, 10, 11, 12, 13, 14, respectively. In particular, the nucleic acid molecule of the present invention is substantially identical to a nucleic acid molecule comprising SEQ ID NOS: 3, 7 and 8 or its reverse complement.

As defined herein, “substantially identical” means it has at least 80% identity to said region and/or sequence, It may also have 85%, 90%, 95%, 97%, 98%, and 99% identity. The length of sequence identity comparison may be over a stretch of at least about nine nucleotides, usually at least about 20 nucleotides, more usually at least about 24 nucleotides. As a practical matter, whether any particular nucleic acid molecule is at least 80%, 85%, 90%, 95%, 96%, 97%, 98% or 99% identical to a nucleotide sequence of the presence invention can be determined conventionally using known computer programs, particularly, the GAP program of the GCG Wisconsin Package (Genetics Computer Group, Madison, Wis.) version 10.0 (update January 1999). The GAP program uses the algorithm of Needleman and Wunsch (J. Mol. Biol., 48, 443-453 (1970) to find the alignment of two complete sequences that maximizes the number of matches and minimizes the number of gaps. Preferably, the default values for all GAP search parameters are used, including scoring matrix=BLOSUM62.cmp, gap weight=8, length weight=2, average match=2.912, and average mismatch=−2.003. Alternatively, the nucleic acid molecule of the present invention may be hybridized under stringent conditions.

The availability of probes for CV, including antigens and antibodies, and polynucleotides derived from the genome from which sequences are derived also allows for the development of tissue culture systems which will be of major use in elucidating the biology of CV. This in turn, will lead to the development of new treatment regimens based upon antiviral compounds which preferentially inhibit the replication of, or infection by CV.

Production of Polypeptides

The invention further relates to CV polypeptides having at least about 90% identity to a polypeptide comprising SEQ ID NOS: 9, 10, 11, 12, 13, or 14. Percent identity is generally determined by aligning the residues of the two amino acid sequences (i.e., a candidate amino acid sequence and the amino acid sequence of one of SEQ ID NOs:9-14) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence is the amino acid sequence being compared to an amino acid sequence present in a preferred polypeptide of the present invention. Preferably, two amino acid sequences are compared using the GAP program of the GCG Wisconsin Package (Genetics Computer Group, Madison, Wis.) version 10.0 (update January 1999). The GAP program uses the algorithm of Needleman and Wunsch (J. Mol. Biol., 48, 443-453 (1970) to find the alignment of two complete sequences that maximizes the number of matches and minimizes the number of gaps. Preferably, the default values for all GAP search parameters are used, including scoring matrix=BLOSUM62.cmp, gap weight=8, length weight=2, average match=2.912, and average mismatch=−2.003. In the comparison of two amino acid sequences using the GAP search algorithm, structural similarity is referred to as “percent identity.” Preferably, a polypeptide includes an amino acid sequence having a structural similarity with SEQ ID NO:9, 10, 11, 12, 13 or 14 of at least about 90 or 95%, more preferably at least about 97%, most preferably at least about 99% identity.

The invention is further directed to a nucleic acid construct comprising expression control sequences and nucleic acid molecules of the present invention.

Alternatively, if the isolated nucleic acid of the present invention is an RNA sequence, a cDNA sequence may be obtained and used to produce a recombinant Chris-like virus comprising a heterologous sequence using methods known in the art. The heterologous sequence might be expressed as an additional transcription unit containing viral gene start and gene end sequences and would be placed between any two CV genes such as between the P and M genes or between the M and F genes. Alternatively, the “foreign” sequence could be expressed as a fusion with a viral protein or expressed as part of a bicistronic mRNA produced from the viral genome.

The invention is also directed to a nucleic acid molecule containing Chris-like virus expression control sequences and a heterologous nucleic acid sequence, as well as a vector and host comprising said sequence.

The nucleic acid sequence encoding the desired polypeptide, whether in fused or mature form, and whether or not containing a signal sequence to permit secretion, may be ligated into expression vectors suitable for any convenient host. Both eukaryotic and prokaryotic host systems are presently used in forming recombinant polypeptides. The polypeptide is then isolated from lysed cells or from the culture medium and purified to the extent needed for its intended use. Purification may be by techniques known in the art, for example, differential extraction, salt fractionation, chromatography on ion exchange resins, affinity chromatography, centrifugation, and the like. See, for example, Methods in Enzymology for a variety of methods for purifying proteins. Such polypeptides can be used as diagnostics, or those, which give rise to neutralizing antibodies, may be formulated into vaccines. Both prokaryotic and eukaryotic host cells may be used for expression of desired coding sequences when appropriate control sequences, which are compatible with the designated host, are used. Among prokaryotic hosts, E. coli is most frequently used. Expression control sequences for prokaryotes include promoters, optionally containing operator portions, and ribosome binding sites. Transfer vectors compatible with prokaryotic hosts are commonly derived from, for example, pBR322, a plasmid containing operons conferring ampicillin and tetracycline resistance, and the various pUC vectors, which also contain sequences conferring antibiotic resistance markers. These markers may be used to obtain successful transformants by selection. Commonly used prokaryotic control sequences include the Beta-lactamase (penicillinase) and lactose promoter systems (Chang et al. (1977)), the tryptophan (trp) promoter system (Goeddel et al. (1980)) and the lambda-derived P_{L} promoter and N gene ribosome binding site (Shimatake et al. (1981)) and the hybrid tac promoter (De Boer et al., (1983)) derived from sequences of the trp and lac UV5 promoters. The foregoing systems are particularly compatible with E. coli; if desired, other prokaryotic hosts such as strains of Bacillus or Pseudomonas may be used, with corresponding control sequences.

Eukaryotic hosts include yeast and mammalian cells in culture systems. Saccharomyces cerevisiae and Saccharomyces carlsbergensis are the most commonly used yeast hosts, and are convenient fungal hosts. Yeast compatible vectors carry markers that permit selection of successful transformants by conferring prototrophy to auxotrophic mutants or resistance to heavy metals on wild-type strains. Yeast compatible vectors may employ the 2 micron origin of replication (Broach et al. (1983)), the combination of CEN3 and ARS1 or other means for assuring replication, such as sequences which will result in incorporation of an appropriate fragment into the host cell genome. Control sequences for yeast vectors are known in the art and include promoters for the synthesis of glycolytic enzymes (Hess et al. (1968); Holland et al. (1978)), including the promoter for 3 phosphoglycerate kinase (Hitzeman (1980)). Terminators may also be included, such as those derived from the enolase gene (Holland (1981)). Particularly useful control systems are those which comprise the glyceraldehyde-3 phosphate dehydrogenase (GAPDH) promoter or alcohol dehydrogenase (ADH) regulatable promoter, terminators also derived from GAPDH, and if secretion is desired, leader sequence from yeast alpha factor. In addition, the transcriptional regulatory region and the transcriptional initiation region that are operably linked may be such that they are not naturally associated in the wild-type organism. These systems are described in detail in EPO 120,551, published Oct. 3, 1984; EPO 116,201, published Aug. 22, 1984; and EPO 164,556, published Dec. 18, 1985, all of which are hereby incorporated herein by reference.

Mammalian cell lines available as hosts for expression are known in the art and include many immortalized cell lines available from the American Type Culture Collection (ATCC), including HeLa cells, Chinese hamster ovary (CHO) cells, baby hamster kidney (BHK) cells, and a number of other cell lines. Suitable promoters for mammalian cells are also known in the art and include viral promoters such as that from Simian Virus 40 (SV40) (Fiers (1978)), Rous sarcoma virus (RSV), adenovirus (ADV), and bovine papilloma virus (BPV). Mammalian cells may also require terminator sequences and poly A addition sequences; enhancer sequences which increase expression may also be included, and sequences which cause amplification of the gene may also be desirable. These sequences are known in the art.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. For example, in selecting a vector, the host must be considered because the vector must function in it. The vector's copy number, the ability to control that copy number, and the expression of any other proteins encoded by the vector, such as antibiotic markers, will also be considered.

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence/host combinations that will express the nucleic acid molecules of this invention on fermentation or in large scale animal culture.

It is further intended that CV protein analogs may be prepared from nucleotide sequences of the protein complex/subunit derived within the scope of the present invention. Analogs, such as fragments, may be produced, for example, by pepsin digestion of CV material. Other analogs, such as muteins, can be produced by standard site-directed mutagenesis of CV protein coding sequences. Analogs exhibiting “CV activity” such as small molecules, whether functioning as promoters or inhibitors, may be identified by known in vivo and/or in vitro assays.

As mentioned above, a DNA sequence encoding a CV protein(s) can be prepared synthetically rather than cloned. The DNA sequence can be designed with the appropriate codons for the CV protein amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984).

Synthetic DNA sequences allow convenient construction of genes which will express CV analogs or “muteins”. Alternatively, DNA encoding muteins can be made by site-directed mutagenesis of native CV genes, RNA(s) or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

The CV antigens may also be isolated from CV virions. The virions may be grown in CV infected cells in tissue culture, or in an infected host.

Preparation of Antigenic Polypeptides and Conjugation with Carrier

An antigenic region of a polypeptide is generally relatively small—typically 8 to 10 amino acids or less in length. Fragments of as few as 5 amino acids may characterize an antigenic region. These segments may correspond to regions of CV antigen. Accordingly, using the sequences of CV as a basis, DNAs encoding short segments of CV polypeptides can be expressed recombinantly either as fusion proteins, or as isolated polypeptides. In addition, short amino acid sequences can be conveniently obtained by chemical synthesis. In instances wherein the synthesized polypeptide is correctly configured so as to provide the correct epitope, but is too small to be immunogenic, the polypeptide may be linked to a suitable carrier.

A number of techniques for obtaining such linkage are known in the art, including the formation of disulfide linkages using N-succinimidyl-3-(2-pyridylthio)propionate (SPDP) and succinimidyl 4-(N-maleimidomethyl)cyclohexane-1-carboxylate (SMCC) obtained from Pierce Company, Rockford, Ill., (if the peptide lacks a sulfhydryl group, this can be provided by addition of a cysteine residue.) These reagents create a disulfide linkage between themselves and peptide cysteine residues on one protein and an amide linkage through the epsilonamino on a lysine, or other free amino group in the other. A variety of such disulfide/amide-forming agents are known. See, for example, Immun. Rev. (1982) 62:185. Other bifunctional coupling agents form a thioether rather than a disulfide linkage. Many of these thio-ether-forming agents are commercially available and include reactive esters of 6-maleimidocaproic acid, 2-bromoacetic acid, 2-iodoacetic acid, 4-(N-maleimidomethyl)cyclohexane-1-carboxylic acid, and the like. The carboxyl groups can be activated by combining them with succinimide or 1-hydroxyl-2-nitro-4-sulfonic acid, sodium salt. Additional methods of coupling antigens employs the rotavirus/“binding peptide” system described in EPO Pub. No. 259,149, the disclosure of which is incorporated herein by reference. The foregoing list is not meant to be exhaustive, and modifications of the named compounds can clearly be used.

Any carrier may be used which does not itself induce the production of antibodies harmful to the host. Suitable carriers are typically large, slowly metabolized macromolecules such as proteins; polysaccharides, such as latex functionalized sepharose, agarose, cellulose, cellulose beads and the like; polymeric amino acids, such as polyglutamic acid, polylysine, and the like; amino acid copolymers; and inactive virus particles. Especially useful protein substrates are serum albumins, keyhole limpet hemocyanin, immunoglobulin molecules, thyroglobulin, ovalbumin, tetanus toxoid, and other proteins well known to those skilled in the art.

In addition to full-length viral proteins, polypeptides comprising truncated CV amino acid sequences encoding at least one viral epitope are useful immunological reagents. For example, polypeptides comprising such truncated sequences can be used as reagents in an immunoassay. These polypeptides also are candidate subunit antigens in compositions for antiserum production or vaccines. While these truncated sequences can be produced by various known treatments of native viral protein, it is generally preferred to make synthetic or recombinant polypeptides comprising an CV sequence. Polypeptides comprising these truncated CV sequences can be made up entirely of CV sequences (one or more epitopes, either contiguous or noncontiguous), or CV sequences and heterologous sequences in a fusion protein. Useful heterologous sequences include sequences that provide for secretion from a recombinant host, enhance the immunological reactivity of the CV epitope(s), or facilitate the coupling of the polypeptide to an immunoassay support or a vaccine carrier. See, e.g., EPO Pub. No. 116,201; U.S. Pat. No. 4,722,840; EPO Pub. No. 259,149; U.S. Pat. No. 4,629,783, the disclosures of which are incorporated herein by reference.

The size of polypeptides comprising the truncated CV sequences can vary widely, the minimum size being a sequence of sufficient size to provide a CV epitope, while the maximum size is not critical. For convenience, the maximum size usually is not substantially greater than that required to provide the desired CV epitopes and function(s) of the heterologous sequence, if any. Typically, the truncated CV amino acid sequence will range from about 5 to about 100 amino acids in length. More typically, however, the CV sequence will be a maximum of about 50 amino acids in length, preferably a maximum of about 30 amino acids. It is usually desirable to select CV sequences of at least about 10, 12 or 15 amino acids, up to a maximum of about 20 or 25 amino acids.

Truncated CV amino acid sequences comprising epitopes can be identified in a number of ways. For example, the entire viral protein sequence can be screened by preparing a series of short peptides that together span the entire protein sequence. By starting with, for example, 100 mer polypeptides, it would be routine to test each polypeptide for the presence of epitope(s) showing a desired reactivity, and then testing progressively smaller and overlapping fragments from an identified 100 mer to map the epitope of interest. Screening such peptides in an immunoassay is within the skill of the art. It is also known to carry out a computer analysis of a protein sequence to identify potential epitopes, and then prepare oligopeptides comprising the identified regions for screening. It is appreciated by those of skill in the art that such computer analysis of antigenicity does not always identify an epitope that actually exists, and can also incorrectly identify a region of the protein as containing an epitope.

The immunogenicity of the epitopes of CV may also be enhanced by preparing them in mammalian or yeast systems fused with or assembled with particle-forming proteins such as, for example, that associated with hepatitis B surface antigen. See, e.g., U.S. Pat. No. 4,722,840. Constructs wherein the CV epitope is linked directly to the particle-forming protein coding sequences produce hybrids which are immunogenic with respect to the CV epitope. Thus, particles constructed from particle forming protein which include CV sequences are immunogenic with respect to CV and HBV or other virus. In addition, portions of the particle-forming protein coding sequence may be replaced with codons encoding a CV epitope. In this replacement, regions which are not required to mediate the aggregation of the units to form immunogenic particles in yeast or mammals can be deleted, thus eliminating additional HBV antigenic sites from competition with the CV epitope.

Antibodies

The immunogenic polypeptides prepared as described above are used to produce antibodies, including polyclonal and monoclonal. If polyclonal antibodies are desired, a selected animal (e.g., chicken, mouse, rabbit, goat, horse, etc.) is immunized with an immunogenic polypeptide bearing a CV epitope(s). Serum from the immunized animal is collected and treated according to known procedures. If serum containing polyclonal antibodies to a CV epitope contains antibodies to other antigens, the polyclonal antibodies can be purified by immunoaffinity chromatography. Alternatively, polyclonal antibodies may be isolated from a an animal which has been previously infected with CV.

Monoclonal antibodies directed against CV epitopes can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or -transfection with Epstein-Barr virus. See, e.g., U.S. Pat. Nos. 4,341,761; 4,399,121; 4,427,783; 4,444,887; 4,466,917; 4,472,500; 4,491,632; and 4,493,890.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody. Monoclonal antibodies of the present invention can also be constructed by genetic or chromosomal transfection methods, as well as phage display technology, all of which are known in the art. See for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g. Johnson and Chiswell, Current Opinion in Structural Biology 3:5564-571 (1993). Panels of monoclonal antibodies produced against CV epitopes can be screened for various properties; i.e., for isotype, epitope affinity, etc. The general methodology for making monoclonal antibodies by hybridomas is well known.

Antibodies, both monoclonal and polyclonal, which are directed against CV epitopes are particularly useful in diagnosis, and those which are neutralizing are useful in passive immunotherapy. Monoclonal antibodies, in particular, may be used to raise anti-idiotype antibodies.

Anti-idiotype antibodies are immunoglobulins which carry an “internal image” of the antigen of the infectious agent against which protection is desired. These anti-idiotype antibodies may also be useful for treatment, vaccination and/or diagnosis of CV infection, as well as for an elucidation of the immunogenic regions of CV antigens.

Vaccines

Vaccines may be prepared from one or more immunogenic polypeptides derived from CV. The observed homology between CV and paramyxoviruses provides information concerning the polypeptides which are likely to be most effective as vaccines, as well as the regions of the genome in which they are encoded. Thus, vaccines may be comprised of recombinant polypeptides containing epitopes of CV protein, including particularly the F protein. These polypeptides may be expressed in various host cells (e.g., bacteria, yeast, insect, or mammalian cells), or alternatively may be isolated from viral preparations. It is also anticipated that the other structural proteins may also contain epitopes which give rise to protective anti-CV antibodies. Thus, polypeptides containing the epitopes of P, C, V and W may also be used, whether singly or in combination, in CV vaccines. Polypeptides containing M epitopes may also be used, whether singly or in combination, in CV vaccines.

Multivalent vaccines against CV may be comprised of one or more epitopes from one or more structural proteins, and/or one or more epitopes from one or more nonstructural proteins. These vaccines may be comprised of, for example, recombinant CV polypeptides and/or polypeptides isolated from the virions. In addition, it may be possible to use inactivated CV in vaccines; inactivation may be by the preparation of viral lysates, or by other means known in the art to cause inactivation of paramyxoviruses, for example, treatment with organic solvents or detergents, or treatment with formalin.

Moreover, vaccines may also be prepared from attenuated CV strains. The preparation of attenuated CV strains can be undertaken by the skilled artisan based on their own knowledge and the recognition of approaches utilized in other paramyxoviruses. Attenuated strains are isolatable after multiple passages in cell culture and/or an animal model. Detection of an attenuated strain in an infected cell or individual is achievable by techniques known in the art, and could include, for example, the use of antibodies to one or more epitopes encoded in CV as a probe or the use of a polynucleotide containing a CV sequence of at least about 8 nucleotides as a probe.

In addition to the above, it is also possible to prepare live vaccines of attenuated microorganisms which express one or more recombinant CV polypeptides. Alternatively, or in addition, an attenuated strain may be constructed utilizing the genomic information of CV provided herein, and utilizing recombinant techniques. Generally, one would attempt to delete a region of the genome encoding, for example, a polypeptide related to pathogenicity, for instance the anti-interferon protein V protein, but which allows viral replication. In addition, the genome construction would allow the expression of an epitope which gives rise to neutralizing antibodies for CV. The altered genome could then be utilized to transform cells which allow CV replication, and the cells grown under conditions to allow viral replication. Attenuated CV strains are useful not only for vaccine purposes, but also as sources for the commercial production of viral antigens, since the processing of these viruses would require less stringent protection measures for the employees involved in viral production and/or the production of viral products.

It is known that some of the proteins in paramyxoviruses contain highly conserved regions. Thus, some immunological cross-reactivity is possible between CV and other paramyxoviruses. It is possible that shared epitopes between the paramyxoviruses and CV will give rise to protective antibodies against one or more of the disorders caused by these pathogenic agents. Thus, it may be possible to design multipurpose vaccines based upon this knowledge.

The preparation of vaccines which contain an immunogenic polypeptide(s) as active ingredients, is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the protein encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-Disoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against an immunogenic polypeptide containing an CV antigenic sequence resulting from administration of this polypeptide in vaccines which are also comprised of the various adjuvants.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25%-70%.

The proteins of the invention may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be prophylactically and/or therapeutically effective. The quantity to be administered, which is generally in the range of 5 micrograms to 250 micrograms of antigen per dose, depends on the subject to be treated, capacity of the subject's immune system to synthesize antibodies, and the degree of protection desired. Precise amounts of active ingredient required to be administered may depend on the judgment of the practitioner and may be peculiar to each subject.

The vaccine may be given in a single dose schedule, or preferably in a multiple dose schedule. A multiple dose schedule is one in which a primary course of vaccination may be with 1-10 separate doses, followed by other doses given at subsequent time intervals required to maintain and or reinforce the immune response, for example, at 1-4 months for a second dose, and if needed, a subsequent dose(s) after several months. The dosage regimen will also, at least in part, be determined by the need of the individual and be dependent upon the judgment of the practitioner.

In addition, the vaccine containing the immunogenic CV antigen(s) may be administered in conjunction with other immunoregulatory agents, for example, immune globulins or immunomodulatory molecules and factors.

Antiviral Agents

The invention is further directed to an agent that modulates the production or expression of the nucleic acid molecule of the present invention and/or modulates replication of CV virus and includes, for example, those which interact with virion components and/or cellular components which are necessary for the binding and/or replication of the virus, and those which inhibit the function of the viral fusion protein. Typical anti-viral agents may include, for example, inhibitors of virion polymerase and/or protease(s) necessary for cleavage of the precursor polypeptides. These may be peptide inhibitors (about 5-20 amino acids) that bind the virion polymerase. Alternatively, the antiviral agents may bind the F protein and may be peptide or peptide analogs between 5 to about 20 amino acids. These peptides might function to inhibit fusion protein function(s), for example they might target its membrane fusion/viral entry function. Other anti-viral agents may include those which act with nucleic acids to prevent viral replication, for example, anti-sense polynucleotides, etc.

Oligomers of about fifteen nucleotides and molecules that hybridize to the AUG initiation codon will be particularly efficient, since they are easy to synthesize and are likely to pose fewer problems than larger molecules when introducing them into CV-producing cells. Antisense methods have been used to inhibit the expression of many genes in vitro (Marcus-Sekura, 1988; Hambor et al., 1988). Antisense polynucleotides molecules are comprised of a complementary nucleotide sequence which allows them to hybridize specifically to designated regions of genomes or RNAs. Antisense polynucleotides may include, for example, molecules that will block protein translation by binding to mRNA, or may be molecules that prevent replication of viral RNA by transcriptase. They may also include molecules that carry agents (non-covalently attached or covalently bound) which cause the viral RNA to be inactive by causing, for example, scissions in the viral RNA. They may also bind to cellular polynucleotides which enhance and/or are required for viral infectivity, replicative ability, or chronicity. Antisense molecules which are to hybridize to CV derived RNAs may be designed based upon the sequence information of the CV sequences provided herein. The antiviral agents based upon anti-sense polynucleotides for CV may be designed to bind with high specificity, to be of increased solubility, to be stable, and to have low toxicity. Hence, they may be delivered in specialized systems, for example, liposomes, or by gene therapy. In addition, they may include analogs, attached proteins, substituted or altered bonding between bases, etc.

The agent may also be a ribozyme. Ribozymes are RNA-protein complexes that cleave nucleic acids in the site-specific fashion. A ribozyme targets the RNA genome and RNA transcripts and copies thereof. Each ribozyme molecule contains a catalytically active segment capable of cleaving the plus or minus strand of the viral RNA, and further comprises flanking sequences having a nucleotide sequence complementary to portions of the target RNA. The flanking sequences serve to anneal the ribozyme to the RNA in a site-specific manner. Absolute complementarity of the flanking sequences to the target sequence is not necessary, however, as only an amount of complementarity sufficient to form a duplex with the target RNA and to allow the catalytically active segment of the ribozyme to cleave at the target sites is necessary. Thus, only sufficient complementarity to permit the ribozyme to be hybridizable with the target RNA is required. In preferred embodiments of the present invention the enzymatic RNA molecule is formed in a hammerhead motif but the ribozyme may also be formed in the motif of a hairpin, hepatitis delta virus, group I intron or RNAse P RNA (in association with an RNA guide sequence). Examples of hammerhead motifs are described by Rossi et al., AIDS Res. Hum. Retrovir. 8:183 (1992), hairpin motifs are described by Hampel et al., Biochem. 28:4929 (1989) and Hampel et al., Nucl. Acids Res. 18:299 (1990), the hepatitis delta virus motif is exemplified in Perrotta and Been, Biochem. 31:16 (1992), an RNAseP motif is described in Gueerier-Takada et al., Cell 35:849 (1983), and examples of the group I intron motif are described in Cech et al., U.S. Pat. No. 4,987,071, each of the foregoing disclosures being incorporated herein by reference.

The use of RNA interference strategies to inhibit the expression of CV protein(s) is further embodied in the invention. Thus, methods of RNA interference and small interfering RNA compositions are included in the methods and compositions of the present invention. RNA interference refers to the silencing of genes specifically by double stranded RNA (dsRNA) (Fine, A. et al (1998) Nature 391;806-811). In one embodiment, short or small interfering RNA (siRNA) is utilized (Elbashir, S. M. et al (2001) Nature 411:494-498). In addition, long double stranded RNA hairpins may be employed (Tavernarakis, N. et al (2000) Nature Genet 24:180-183; Chuang, C. F. and Meyerowitz, E. M. (2000) PNAS USA 97:4985-90; Smith, N A et al (2000) Nature 407:319-20). Virus-mediated RNA interference against K-Ras has been described (B rummelkamp, T. R. et al (2002) Cancer Cell 2:243-247).

Other types of drugs may be based upon polynucleotides which “mimic” important control regions of the CV genome, and which may be therapeutic due to their interactions with key components of the system responsible for viral infectivity or replication.

Compositions

The present invention further contemplates therapeutic/pharmaceutical compositions useful in practicing the therapeutic methods of this invention. The terms “thereapeutic composition” and “pharmaceutical composition” will be used interchangeably. A subject therapeutic composition includes, in admixture, a pharmaceutically acceptable excipient (carrier) and one or more of a CV protein, polypeptide analog thereof or fragment thereof, as described herein as an active ingredient. In a preferred embodiment, the composition comprises a CV antigen, antiviral agent or immunogenic CV polypeptide. The preparation of therapeutic compositions which contain polypeptides, analogs or active fragments as active ingredients is well understood in the art. Compositions comprising the antibodies of the present invention could be used for prophylaxis and may be formulated into a vaccine formulation described infra.

Compositions comprising antiviral agents, particularly antisense oligonucleotides, ribozymes and/or siRNAs may be introduced directly to the subject using methods known in the art (see, for example, Rolland, 1998, Crit. Rev. Therap. Drug Carrier Systems 15:143-198 and references cited therein and Ulmer, 1993, Science 259:1745-1749). The uptake of naked polynucleotide may be increased by coating the polynucleotide onto biodegradable beads, which are efficiently transported into the cells. Other methods to directly introduce the agents of the present invention into cells or exploit receptors on the surface of cells include but are not limited to the use of liposomes and lipids, ligands for specific cell surface receptors, cell receptors, and calcium phosphate and other chemical mediators, microinjections directly to single cells, electroporation and homologous recombination. Liposomes are commercially available from Invitrogen, for example, as LIPOFECTIN^{•} and LIPOFECTACE^{•}, which are formed of cationic lipids such as N-[1-(2,3 dioleyloxy)-propyl]-n,n,n-trimethylammonium chloride (DOTMA) and dimethyl dioctadecylammonium bromide (DDAB). Numerous methods are also published for making liposomes, known to those skilled in the art.

For example, nucleic acid-lipid complexes—lipid carriers can be associated with naked nucleic acids (e.g., plasmid DNA) to facilitate passage through cellular membranes. Cationic, anionic, or neutral lipids can be used for this purpose. However, cationic lipids are preferred because they have been shown to associate better with DNA which, generally, has a negative charge. Cationic lipids have also been shown to mediate intracellular delivery of plasmid DNA (Felgner and Ringold, Nature 337:387 (1989)). Intravenous injection of cationic lipid-plasmid complexes into mice has been shown to result in expression of the DNA in lung (Brigham et al., Am. J. Med. Sci. 298:278 (1989)). See also, Osaka et al., J. Pharm. Sci. 85(6):612-618 (1996); San et al., Human Gene Therapy 4:781-788 (1993); Senior et al., Biochemica et Biophysica Acta 1070:173-179 (1991); Kabanov and Kabanov, Bioconjugate Chem. 6:7-20 (1995); Remy et al., Bioconjugate Chem. 5:647-654 (1994); Behr, J-P., Bioconjugate Chem 5:382-389 (1994); Behr et al., Proc. Natl. Acad. Sci., USA 86:6982-6986 (1989); and Wyman et al., Biochem. 36:3008-3017 (1997).

Cationic lipids are known to those of ordinary skill in the art. Representative cationic lipids include those disclosed, for example, in U.S. Pat. No. 5,283,185; and e.g., U.S. Pat. No. 5,767,099. In a preferred embodiment, the cationic lipid is N.sup.4-spermine cholesteryl carbamate (GL-67) disclosed in U.S. Pat. No. 5,767,099. Additional preferred lipids include N4_spermidine cholestryl carbamate (GL-53) and 1-(N-4-spermind)-2,3-dilaurylglycerol carbamate (GL-89).

Formulation of the compositions of the present invention will depend upon the route chosen for administration. The compositions utilized in this invention can be administered by various routes including both enteral and parenteral routes, including oral, intravenous, intramuscular, subcutaneous, inhalation, intrathecal, intraventricular, transmucosal, transdermal, intranasal, intraperitoneal, and intrapulmonary. The composition may comprise one or more agents of the present invention.

Oral dosage forms can be formulated as tablets, pills, dragees, capsules, liquids, gels, syrups, slurries, suspensions, and the like, for ingestion by the patient. Solid formulations of the compositions for oral administration can contain suitable carriers or excipients, such as carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; and other agents such as acacia and alginic acid. Agents that facilitate disintegration and/or solubilization can be added, such as the cross-linked polyvinyl pyrrolidone, agar, alginic acid, or a salt thereof, such as sodium alginate, microcrystalline cellulose, corn starch, sodium starch glycolate, and alginic acid. Tablet binders that can be used include acacia, methylcellulose, carboxymethylcellulose, polyvinylpyrrolidone (Povidon™), hydroxypropyl methylcellulose, sucrose, starch and ethylcellulose. Lubricants that can be used include magnesium stearates, stearic acid, silicone fluid, talc, waxes, oils, and colloidal silica. Fillers, agents that facilitate disintegration and/or solubilization, tablet binders and lubricants, including the aforementioned, can be used singly or in combination. Solid oral dosage forms need not be uniform throughout. For example, dragee cores can be used in conjunction with suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures.

Liquid formulations of the pharmaceutical compositions for oral (enteral) administration are prepared in water or other aqueous vehicles and can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents.

The pharmaceutical or therapeutic compositions of the present invention can also be formulated for parenteral administration. Formulations for parenteral administration can be in the form of aqueous or non-aqueous isotonic sterile injection solutions or suspensions. Parenteral formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like).

For intravenous injection, water soluble versions of the compounds of the present invention are formulated in, or if provided as a lyophilate, mixed with, a physiologically acceptable fluid vehicle, such as 5% dextrose (“D5”), physiologically buffered saline, 0.9% saline, Hanks' solution, or Ringer's solution. Intravenous formulations may include carriers, excipients or stabilizers including, without limitation, calcium, human serum albumin, citrate, acetate, calcium chloride, carbonate, and other salts.

Intramuscular preparations, e.g. a sterile formulation of a suitable soluble salt form of the compounds of the present invention, can be dissolved and administered in a pharmaceutical excipient such as Water-for-Injection, 0.9% saline, or 5% glucose solution. Alternatively, a suitable insoluble form of the compound can be prepared and administered as a suspension in an aqueous base or a pharmaceutically acceptable oil base, such as an ester of a long chain fatty acid (e.g., ethyl oleate), fatty oils such as sesame oil, triglycerides, or liposomes.

Aqueous injection suspensions can also contain substances that increase the viscosity of the suspension, such as sodium carboxymethyl cellulose, sorbitol, or dextran. Non-lipid polycationic amino polymers can also be used for delivery. Optionally, the suspension can also contain suitable stabilizers or agents that increase the solubility of the compounds to allow for the preparation of highly concentrated solutions.

Pharmaceutical or therapeutic compositions of the present invention can also be formulated to permit injectable, long-term, deposition. Injectable depot forms may be made by forming microencapsulated matrices of the compound in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in microemulsions that are compatible with body tissues.

Inhalation formulations can also readily be formulated. For inhalation, various powder and liquid formulations can be prepared. For aerosol preparations, a sterile formulation of the compound or salt form of the compound may be used in inhalers, such as metered dose inhalers, and nebulizers. Aerosolized forms may be especially useful for treating respiratory disorders.

Alternatively, the compounds of the present invention can be in powder form for reconstitution in the appropriate pharmaceutically acceptable carrier at the time of delivery. The pharmaceutically active compound in the pharmaceutical compositions of the present invention can be provided as the salt of a variety of acids. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

After pharmaceutical compositions have been prepared, they are packaged in an appropriate container and labeled for treatment of an indicated condition.

The active compound will be present in an amount effective to achieve the intended purpose. The determination of an effective dose is well within the capability of those skilled in the art. The therapeutically effective dose of the pharmaceutical agents of the present invention can be estimated initially by in vitro tests, such as cell culture assays, followed by assay in model animals, usually mice, rats, rabbits, dogs, or pigs. The animal model can also be used to determine an initial preferred concentration range and route of administration. For example, the ED50 (the dose therapeutically effective in 50% of the population) and LD50 (the dose lethal to 50% of the population) can be determined in one or more cell culture of animal model systems. The dose ratio of toxic to therapeutic effects is the therapeutic index, which can be expressed as LD50/ED50. Pharmaceutical compositions that exhibit large therapeutic indices are preferred.

The data obtained from cell culture assays and animal studies are used in formulating an initial dosage range for human use, and preferably provide a range of circulating concentrations that includes the ED50 with little or no toxicity. After administration, or between successive administrations, the circulating concentration of active agent varies within this range depending upon pharmacokinetic factors well known in the art, such as the dosage form employed, sensitivity of the patient, and the route of administration.

The exact dosage will be determined by the practitioner, in light of factors specific to the subject requiring treatment. Factors that can be taken into account by the practitioner include the severity of the disease state, general health of the subject, age, weight, gender of the subject, diet, time and frequency of administration, drug combination(s), reaction sensitivities, and tolerance/response to therapy. Long-acting pharmaceutical compositions can be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Where the therapeutic agent is a protein or antibody of the present invention, the therapeutic protein or antibody agent typically is administered at a daily dosage of 0.01 mg to 30 mg/kg of body weight of the patient. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The compositions of the present invention may be used to modulate replication of a paramyxovirus, particularly CV virus. In a particular embodiment, the compositions and compounds disclosed may be used to inhibit production or expression of a nucleic acid comprising SEQ ID NOS: 3, 7 and 8.

Compositions comprising the attenuated viruses of the present invention may be used to modulate tumor growth in a subject. The subject may be an animal or a human subject. The tumor may be a sarcoma, melanoma, breast carcinoma, ovarian carcinoma, bladder carcinoma, colon carcinoma, prostate carcinoma, small cell and non-small cell lung carcinomas, or glioblastoma.

Diagnostics

Using the disclosed CV sequences, oligomers of approximately 8 nucleotides or more can be prepared, either by excision from recombinant polynucleotides or synthetically, which hybridize with the CV genome and are useful in identification of the viral agent(s), further characterization of the viral genome(s), as well as in detection of the virus(es) in diseased individuals. The CV polynucleotide (natural or derived) probes or primers are a length which allows the detection of unique viral sequences by hybridization or amplification. While 6-8 nucleotides may be a workable length, sequences of 10-12 nucleotides are preferred, and about 20 nucleotides appears optimal. Preferably, these sequences will derive from regions which lack heterogeneity. These probes can be prepared using routine methods, including automated oligonucleotide synthetic methods. A complement to any unique portion of the CV genome will be satisfactory. For use as probes, complete complementarity is desirable, though it may be unnecessary as the length of the fragment is increased.

For use of such probes as diagnostics, the biological sample to be analyzed, such as blood or serum, may be treated, if desired, to extract the nucleic acids contained therein. The resulting nucleic acid from the sample may be subjected to gel electrophoresis or other size separation techniques; alternatively, the nucleic acid sample may be dot blotted without size separation. The probes are usually labeled. Suitable labels, and methods for labeling probes are known in the art, and include, for example, radioactive labels incorporated by nick translation or kinasing, biotin, fluorescent probes, and chemiluminescent probes. The nucleic acids extracted from the sample are then treated with the labeled probe under hybridization conditions of suitable stringencies.

The probes or primers can be made completely complementary to the CV genome. Therefore, usually high stringency conditions are desirable in order to prevent false positives. The stringency of hybridization is determined by a number of factors during hybridization and during the washing procedure, including temperature, ionic strength, length of time, and concentration of formamide. These factors are outlined in, for example, Sambrook et al., Molecular Cloning: A Laboratory Manual, 3d ed., Cold Spring Harbor Press (2001).

The CV genome sequences may be present in serum or other biological fluid or sample of infected individuals at relatively low levels and may require that amplification techniques be used in hybridization assays. Methods of performing primer-directed amplification are also well-known in the art using the above-described primers; see, for example, McPherson, PCR Basics: From Background to Bench, Springer Verlag (2000); Innis et al. (eds.), PCR Applications: Protocols for Functional Genomics, Academic Press (1999); Gelfand et al. (eds.), PCR Strategies, Academic Press (1998); Newton et al., PCR, Springer-Verlag New York (1997); Burke (ed.), PCR: Essential Techniques, John Wiley & Son Ltd (1996); White (ed.), PCR Cloning Protocols: From Molecular Cloning to Genetic Engineering, Vol. 67, Humana Press (1996); McPherson et al. (eds.), PCR 2: A Practical Approach, Oxford University Press, Inc. (1995). Methods for performing RT-PCR are collected, e.g., in Siebert et al. (eds.), GeneCloning and Analysis by RT-PCR, Eaton Publishing Company/Bio. The Techniques Books Division, 1998; Siebert (ed.), PCR Technique:RT-PCR, Eaton Publishing Company/BioTechniques Books (1995). Amplified nucleic acid may be detected using methods known in the art, by, for example detecting the labeled amplified nucleic acid or alternatively by mass spectrometry.

The probes or primers can be packaged into diagnostic kits. Diagnostic kits include the probe DNA, which may be labeled; alternatively, the probe DNA may be unlabeled and the ingredients for labeling may be included in the kit in separate containers. The kit may also contain other suitably packaged reagents and materials needed for the particular hybridization protocol, for example, standards, as well as instructions for conducting the test. The probes may be attached to a solid support. The CV sequences provided herein may be used to gain further information on the sequence of the CV genome, and for identification and isolation of the CV agent, and thus will aid in its characterization including the nature of the genome, the structure of the viral particle, and the nature of the antigens of which it is composed. This information, in turn, can lead to additional polynucleotide probes, polypeptides derived from the CV genome, and antibodies directed against CV epitopes that would be useful for the diagnosis and/or treatment of Chris-like virus infection.

The CV sequence information is useful for the design of probes for the isolation of additional sequences which are derived from as yet undefined regions of the CV genome(s). For example, labeled probes containing a sequence of approximately 8 or more nucleotides, and preferably 20 or more nucleotides, including those derived from regions close to the 5′-termini or 3′-termini of the family of CV sequences may be used to isolate overlapping sequences from CV. Unless the CV genome is segmented and the segments lack common sequences, it is possible to sequence the entire viral genome(s) utilizing the technique of isolation of overlapping cDNAs or RNAs derived from the viral genome(s). Alternatively, characterization of the genomic segments could be from the viral genome(s) isolated from purified CV particles. Procedures for isolating polynucleotide genomes from viral particles are known in the art. The isolated genomic segments could then be cloned and sequenced. Thus, with the information provided herein, it is possible to clone and sequence the CV genome(s) irrespective of their nature.

Both the polypeptides which react immunologically with serum containing CV antibodies, and the antibodies raised against the CV specific epitopes in these polypeptides are useful in immunoassays to detect presence of CV antibodies, or the presence of the virus and/or viral antigens, in biological samples. Design of the immunoassays is subject to a great deal of variation, and many formats are known in the art. The immunoassay will utilize at least one viral epitope derived from CV. In one embodiment, the immunoassay uses a combination of viral epitopes derived from CV. These epitopes may be derived from the same or from different viral polypeptides, and may be in separate recombinant or natural polypeptides, or together in the same recombinant polypeptides. An immunoassay may use, for example, a monoclonal antibody directed towards a viral epitope(s), a combination of monoclonal antibodies directed towards epitopes of one viral antigen, monoclonal antibodies directed towards epitopes of different viral antigens, polyclonal antibodies directed towards the same viral antigen, or polyclonal antibodies directed towards different viral antigens. Preferably, the anti-CV antibody used in the diagnostic methods of this invention is an affinity purified polyclonal antibody. More preferably, the antibody is a monoclonal antibody (mAb). In addition, it is preferable for the anti-CV antibody molecules used herein be in the form of Fab, Fab′, F(ab′)₂ or F(v) portions of whole antibody molecules.

Protocols may be based, for example, upon competition, or direct reaction, or sandwich type assays. Protocols may also, for example, use solid supports, or may be by immunoprecipitation. Most assays involve the use of labeled antibody or polypeptide; the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Typically, an immunoassay for an anti-CV antibody(s) will involve selecting and preparing the test sample suspected of containing the antibodies, such as a biological sample, then incubating it with an antigenic (i.e., epitope-containing) CV polypeptide(s) under conditions that allow antigen-antibody complexes to form, and then detecting the formation of such complexes. Suitable incubation conditions are well known in the art. The immunoassay may be, without limitations, in a heterogeneous or in a homogeneous format, and of a standard or competitive type.

Complexes formed comprising anti-CV antibody (or, in the case of competitive assays, the amount of competing antibody) are detected by any of a number of known techniques, depending on the format. For example, unlabeled CV antibodies in the complex may be detected using a conjugate of antixenogeneic Ig complexed with a label, (e.g., an enzyme label).

In immunoassays where CV polypeptides are the analyte, the test sample, typically a biological sample, is incubated with anti-CV antibodies under conditions that allow the formation of antigen-antibody complexes. Various formats can be employed. For example, a “sandwich assay” may be employed, where antibody bound to a solid support is incubated with the test sample; washed; incubated with a second, labeled antibody to the analyte, and the support is washed again. Analyte is detected by determining if the second antibody is bound to the support. In a competitive format, which can be either heterogeneous or homogeneous, a test sample is usually incubated with antibody and a labeled, competing antigen is also incubated, either sequentially or simultaneously. These and other formats are well known in the art.

Kits suitable for immunodiagnosis and containing the appropriate labeled reagents are constructed by packaging the appropriate materials, including the polypeptides of the invention containing CV epitopes or antibodies directed against CV epitopes in suitable containers, along with the remaining reagents and materials required for the conduct of the assay, as well as a suitable set of assay instructions.

In a further embodiment of this invention, commercial test kits may be prepared to determine the presence or absence of CV, determine the presence or absence of predetermined CV protein activity or predetermined CV protein activity capability in suspected target cells. In accordance with the testing techniques discussed above, one class of such kits will contain at least the labeled CV protein(s) or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence of CV in cells or samples, comprising: (a) a predetermined amount of at least one labeled immunochemically reactive component obtained by the direct or indirect attachment of CV protein(s) or a specific binding partner thereto, to a detectable label and optionally (b) other reagents; and (c) directions for use of said kit.

More specifically, the diagnostic test kit may comprise: (a) a known amount of the CV protein(s) or CV as described above (or a binding partner) generally bound to a solid phase to form an immunosorbent, or in the alternative, bound to a suitable tag, or plural such end products, etc. (or their binding partners) one of each and optionally (b) other reagents; and (c) directions for use of said test kit.

In a further variation, the test kit may be prepared and used for the purposes stated above, which operates according to a predetermined protocol (e.g. “competitive,” “sandwich,” “double antibody,” etc.), and comprises: (a) a labeled component which has been obtained by coupling the CV protein(s) to a detectable label; (b) one or more additional immunochemical reagents of which at least one reagent is a ligand or an immobilized ligand, which ligand is selected from the group consisting of (i) a ligand capable of binding with the labeled component (a); (ii) a ligand capable of binding with a binding partner of the labeled component (a); (iii) a ligand capable of binding with at least one of the component(s) to be determined; and (iv) a ligand capable of binding with at least one of the binding partners of at least one of the component(s) to be determined; and (c) directions for the performance of a protocol for the detection and/or determination of one or more components of an immunochemical reaction between the CV protein(s) and a specific binding partner thereto.

The methods and compositions provided herein for detecting CV antigens and CV polynucleotides are useful for screening of anti-viral agents in that they provide an alternative, and perhaps more sensitive means, for detecting the agent's effect on viral replication than the cell plaque assay or ID₅₀ assay. For example, the CV-polynucleotide probes described herein may be used to quantitate the amount of viral nucleic acid produced in a cell culture. This could be accomplished, for example, by hybridization or competition hybridization of the infected cell nucleic acids with a labeled CV-polynucleotide probe.

For example, also, anti-CV antibodies may be used to identify and quantitate CV antigen(s) in the cell culture utilizing the immunoassays described herein. In addition, since it may be desirable to quantitate CV antigens in the infected cell culture by a competition assay, the polypeptides encoded within the CV sequences described herein are useful in these competition assays. Generally, a recombinant CV polypeptide derived from the CV sequence would be labeled, and the inhibition of binding of this labeled polypeptide to a CV polypeptide due to the antigen produced in the cell culture system would be monitored. Moreover, these techniques are particularly useful in cases where the CV may be able to replicate in a cell line without causing cell death. To measure antigen, a known specific antibody is fixed to the solid phase, the test material containing antigen is added, after an incubation the solid phase is washed, and a second enzyme-labeled antibody is added. After washing, substrate is added, and enzyme activity is estimated calorimetrically, and related to antigen concentration.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

Sequences encoding genes of a new and novel paramyxovirus, Chris-like virus (CV) have been identified. Nucleotide sequences identified by Liang et al (Liang, X. et al (2003) J Am Soc Nephrol 14:1443-1451; Liang, X. et al (2003) Hypertens Res. 26(3):225-235) as novel human genes upregulated by angiotensin II (Ang II) in human kidney glomerular mesangial cells are actually RNA from a novel virus. The Liang et al nucleotide sequences are available from the National Center for Biotechnology Information (NCBI) (ncbi.nlm.nih.gov) as accession numbers AF367870, AY040225 and AY032980 (SEQ ID NOS: 1, 3 and 5) (Genbank entry AY032980 was submitted to the database earlier than and is a partial sequence which is encompassed entirely by AY040225, which contains additional 5′ end RNA sequence. Deduced amino acid sequences for AF367870, AY040225 and AY032980 are shown in SEQ ID NOS: 2, 4 and 6 respectively. However, there are several sequence discrepancies within the region of overlap. AY040225 was used for the reconstruction of the M and F protein sequences described below). These sequences, derived by RT-PCR from human mesangial cell RNA stimulated with Ang II, were reported to encode two genes denoted AngRem104 (SEQ ID NO: 1) and AngRem52 (SEQ ID NO: 3). Although reported to encode proteins encoded by cellular genes, we have discovered that these RNAs actually encode viral proteins of a novel paramyxovirus, Chris-like virus (CV). These sequences are partially incorrect as evidenced by the presence of premature stop codons in the sequences. Introduction of nucleotides at points where the apparent inappropriate stop codons appear in the reported sequence or deletion of nucleotides to restore the reading frame, result in the translation of novel, near full-length paramyxovirus viral proteins.

CV is related to but phylogenetically-distinct from Nipah virus, Hendra virus and morbilliviruses, all members of the paramyxovirus family. Nipah and Hendra viruses are highly pathogenic in humans and are pathogens of pigs and horses, respectively. Fifty percent of clinically apparent human cases die. Although members of this group of viruses have only caused a few focal outbreaks, the biologic property of these viruses to infect a wide range of hosts and to produce a disease causing significant mortality in humans has made this emerging viral infection a public heath concern. Morbilliviruses have been identified as pathogens in humans and in animals. Therefore, CV and closely-related viruses are also expected to be human and animal pathogens. The similarity to Nipah and Hendra viruses suggests that CV may be of concern as an “emerging pathogen.” No drug therapies have yet been proven to be effective in treating Nipah infection. Treatment relies on providing intensive supportive care. There is some evidence that early treatment with the antiviral drug, ribavirin, can reduce both the duration of feverish illness and the severity of disease, however, the efficacy of this treatment in curing disease or improving survival is not fully tested.

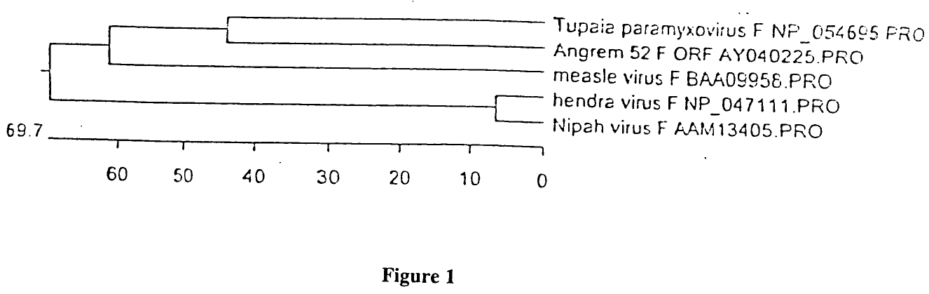

The AY040225 sequence (SEQ ID NO: 3) (and its partial AY032980 sequence (SEQ ID NO:5)) can encode a viral fusion (F) protein (SEQ ID NO: 11). Alignment of the novel F-like open reading frame present in the sequences AY040225 (SEQ ID NO:3) and AY032980 (SEQ ID NO: 5) with the Nipah virus F protein (SEQ ID NO: 17) shows significant sequence similarity. A phylogenetic comparison of F proteins from AY040225 and Nipah virus, Hendra virus, Tupaia paramyxovirus and measles virus, generated by the Clustal Method using the DNAStar Megalign computer program, finds that the reported F protein sequence is distinct from the other F proteins (FIG. 1).

Reconstruction of Putative Chris-Like Virus Open Reading Frames (ORFs) Encoding Phosphoprotein (P), Matrix (M) and Fusion (F) Proteins from the Sequences AF367870 (Angrem104) (SEQ ID NO:1), AY040225 (Angrem52) (SEQ ID NO: 3) and AY032980 (Angrgm-52) (SEQ ID NO: 5)

The AY032980 sequence was submitted to the database approximately 2 months earlier that the sequence AY040225. It appears that AY040225 is a longer version (it contains more sequence from the 5′ end of the mRNA) of the cDNA described by AY032980. However, there are several sequence discrepancies within the region of overlap. AY040225 was used for the reconstruction of the M and F protein sequences described below.

Modification of the Angrem104 Nucleotide Sequence so as to Produce a Sequence Encoding a Phosphoprotein.

The reported Angrem104 sequence is 1690 nucleotides long. The ATG which begins the P ORF is at positions 90-92. A single “T” residue between nucleotides at positions 1130 and 1131 has been inserted. This results in a single reading frame that ends at position 1582 of the reported Angrem104 sequence. The deduced CV P protein was aligned with Nipah Virus P protein (SEQ ID NO:15). The aligned sequences show 20.1% amino acid identity. The modified Angrem104 sequence is presented in SEQ ID NO: 7.

The Matrix Protein Encoding Sequences of Angrem52 (SEQ ID NO:3)

The reported Angrem52 sequence encodes what appears to be a full-length or near full-length paramyxovirus matrix (M) protein. The ORF begins at position 16 and ends at position 1038. The aligned sequences share 51.6% amino acid identity. The Chris-like virus M protein and the measles virus M protein are 44.8% identical. The Nipah virus M protein (SEQ ID NO: 16) and the measles virus M protein are 44.5% identical.

Modification of the Angrem52 Nucleotide Sequence so as to Produce a Sequence Encoding an F Protein

The Angrem52 nucleotide sequence (SEQ ID NO: 8) contains a second ORF which can encode a paramyxovirus F protein. The F ORF begins at position 1393. The reported sequence was modified in several positions as follows. An “A” at position 2110 was deleted. A “T” at position 2155 was deleted. A single nucleotide, either C or T, was added between positions 2296 and 2297. A “T” was deleted at 2461. An alignment of the F protein from the modified Angrem52 sequence to the Nipah virus F protein is provided. The two aligned proteins are 32.4% identical at the amino acid level. The Chris-like virus F protein and the measles virus F protein are 29.5% identical and the Nipah virus F protein (SEQ ID NO: 17) and the measles virus F protein are 31.1% identical.

V, W and C Proteins Encoded by the P Gene of Chris-Like Virus

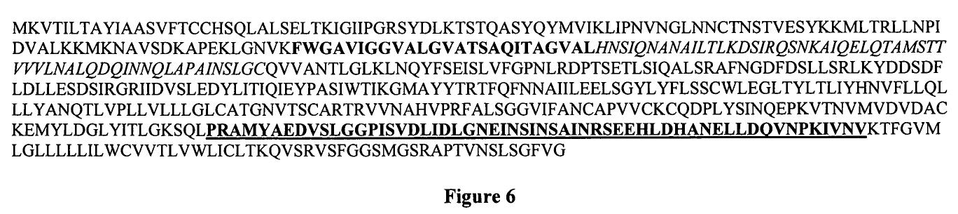

The V protein of Chris-like virus would be encoded by an “edited” (by the viral polymerase) mRNA of the Angrem104 sequence. The editing would involve the addition of a single, non-template encoded “G” at the editing site. The editing site is predicted to be at the sequence 786-AAAAAAGG-793 (of FIG. 6), and the additional G residue would be added to the template encoded Gs in this sequence (i.e. the edited sequence would then be AAAAAAGGG. A “W” protein may also be encoded by the P gene by the addition of two Gs at the editing site. The V ORF would begin at nucleotide 90 of our modified Angrem104 sequence and end at position 967 of the modified angrem104 sequence (this does not count the additional G residue found in the edited mRNA). The carboxy-terminus of this Chris-like virus V protein is cysteine-rich, as expected for a paramyxovirus V protein (SEQ ID NO:12). The predicted protein sequence of the W protein is provided in SEQ ID NO: 14.

A C ORF is found from positions 109-591 of the modified Angrem104 sequence (SEQ ID NO: 7) and encodes a “C” protein (SEQ ID NO:13) within the P coding sequence.

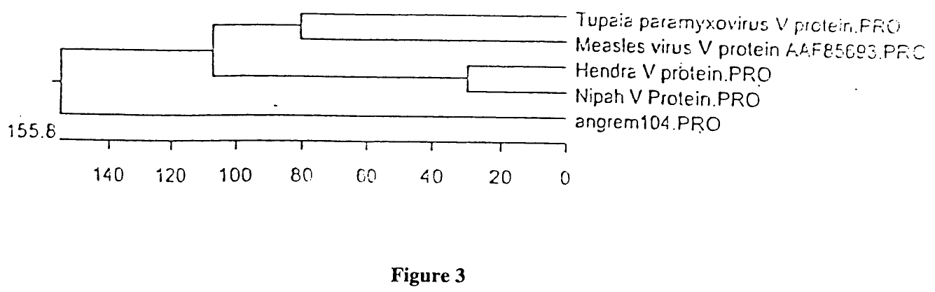

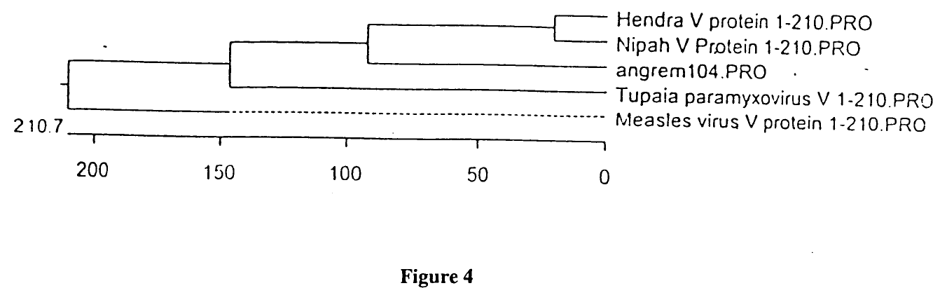

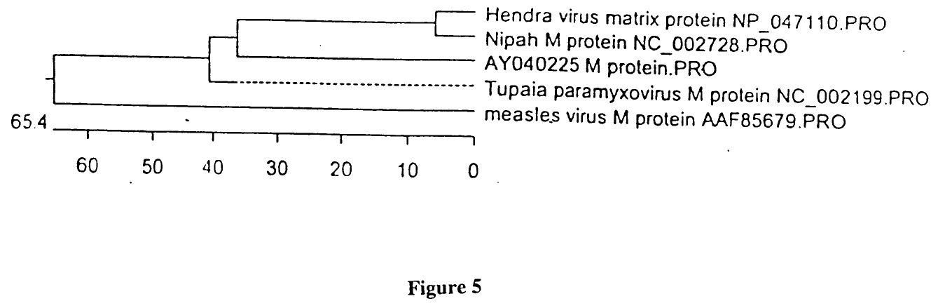

A phylogenetic analysis of the V proteins of the paramyxoviruses and the Chris-like virus, generated by the Clustal method using the DNAStar Megalign computer program, places CV at the root of the tree. Importantly, the V, W and P proteins of Nipah and Hendra virus possess a unique 210 amino-terminal extension absent from other paramyxoviruses (FIG. 3). The P/V-like protein encoded by AF367870 appears to also possess this amino-terminal extension. Comparison of the amino-terminal 210 amino acids of the AF367870 protein with that of the V proteins of Nipah virus, Hendra virus, Tupaia paramyxovirus and measles virus finds that the amino-terminal 210 amino acids of the P/V-like protein encoded by AF367870 are most closely related to the Nipah and Hendra V proteins' amino-termini (FIG. 4). Comparison of the matrix proteins encoded by AY040225 and AY032980 and by Nipah virus, Hendra virus, Tupaia paramyxovirus and measles virus finds that the AY040225 matrix protein is most closely related to Nipah and Hendra viruses (FIG. 5).

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this Specification, each of which is incorporated herein by reference in its entirety.

What is claimed is:

1. An isolated paramyxovirus comprising a nucleic acid molecule hybridizing under stringent conditions to SEQ ID NOS: 3, 7 and 8.

2. An isolated paramyxovirus comprising polypeptides having at least 90% identity to SEQ ID NOS: 9, 10, 11, 12, 13 and 14 using a GAP algorithm with default parameters.

3. An isolated paramyxovirus comprising an identifying characteristic selected from the group consisting of: (a) a nucleic acid molecule hybridizing under stringent conditions to SEQ ID NOS: 3, 7 or 8 a reverse complement thereof; (b) a nucleic acid sequence having at least 90% identity with SEQ ID NOS: 3, 7 or 8 using a GAP algorithm with default parameters or a reverse complement thereof; (c) a nucleic a sequence encoding a polypeptide having amino acid sequences depicted in SEQ ID NOS: 9, 10, 11, 12, 13 or 14 or reverse complement thereof and (c) a polypeptide having at least 90% identify with a polypeptide having amino acid sequences depicted in SEQ ID NOS: 9, 10, 11, 12, 13 or 14 using a GAP algorithm with default parameters.

4. The isolated paramyxovirus of claim 3, wherein said virus comprises a nucleic acid molecule corresponding to SEQ ID NOS: 3, 7 or 8 a reverse complement thereof.

5. The isolated paramyxovirus of claim 3, wherein said virus comprises a polypeptide sequence comprises a polypeptide sequence having an amino acid sequence selected from the group consisting of SEQ ID NOS: 9, 10, 11, 12, 13 and 14.

6. An isolated antibody that binds to an epitope of the isolated paramyxovirus of claim 3.

7. The antibody of claim 6, wherein said antibody is a polyclonal antibody or a monoclonal antibody.

8. An isolated nucleic acid molecule comprising (a) a nucleic acid sequence hybridizing under stringent conditions to a nucleic acid sequence SEQ ID NOS: 3, 7 and 8; (b) a nucleic acid sequence having at least 90% identity to a nucleic acid sequence comprising SEQ ID NOS: 3, 7 and 8; (c) a nucleic acid sequence encoding a polypeptide having at least about 90% identity to a polypeptide comprising SEQ ID NOS: 9, 10, 11, 12, 13 or 14.

9. An isolated nucleic acid molecule comprising SEQ ID NOS: 3, 7 and 8.

10. A nucleic acid construct comprising the isolated nucleic acid molecule of claim 8.

11. A vector comprising the nucleic acid molecule of claim 8.

12. A host cell comprising the nucleic acid molecule of claim 8.

13. An isolated nucleic acid molecule comprising a nucleic acid sequence encoding a polypeptide comprising SEQ ID NOS: 9, 10, 11, 12, 13 or 14.

14. A method for producing a polypeptide comprising SEQ ID NOS: 9, 10, 11, 12, 13 or 14 comprising (a) culturing the host cell of 12 and (b) isolating said polypeptide from said cultured cell.

15. An isolated polypeptide comprising a polypeptide sequence having at least 90% identity to SEQ ID NOS: 9, 10, 11, 12, 13 or 14.

16. The isolated polypeptide according to claim 15 wherein said polypeptide comprises a polypeptide sequence depicted in SEQ ID NOS. 9, 10, 11, 12, 13 or 14.

17. The polypeptide according to claim 16, wherein said polypeptide comprises SEQ ID NO:9.

18. The polypeptide according to claim 17, wherein said polypeptide is encoded by nucleotides 16-1038 of SEQ ID NO:3.

19. The polypeptide according to claim 16, wherein said polypeptide comprises SEQ ID NO:10.

20. The polypeptide according to claim 19, wherein said polypeptide is encoded by nucleotides 90-1582 of SEQ ID NO:7.

21. The polypeptide according to claim 16, wherein said polypeptide comprises SEQ ID NO:11.

22. The polypeptide according to claim 21, wherein said polypeptide is encoded by nucleotides 1393-3039 of SEQ ID NO:8.

23. The polypeptide according to claim 16, wherein said polypeptide comprises SEQ ID NO:12.

24. The polypeptide according to claim 23, wherein said polypeptide is encoded by nucleotides 90-967 of SEQ ID NO:7.

25. The polypeptide according to claim 16, wherein said polypeptide comprises SEQ ID NO:14.

26. The polypeptide according to claim 25, wherein said polypeptide is encoded by nucleotides 109-591 of SEQ ID NO:7.

27. A method for detecting a Chris-like virus comprising contacting a virus particle with the antibody of claim 6 under conditions to form a complex with a virus particle and detecting the complex, wherein the presence of the complex indicates the presence of said virus.

28. The method according to claim 27, wherein said virus particle is obtainable from a biological sample.

29. A method of detecting the presence of absence of a Chris-like virus in a subject, comprising contacting a nucleic acid molecule from a sample from a subject with at least one primer or probe derived from the nucleic acid molecule of claim 8 and detecting the presence or absence of said virus nucleic acid molecule in said sample to detect the presence or absence of said virus.

30. The method according to claim 29, wherein said method further comprises isolating nucleic acid from said subject.

31. A method of detecting the presence or absence of a Chris-like virus nucleic acid sequence in a sample comprising contacting the sample with a probe derived from the nucleic acid molecule of claim 8 under conditions in which the polynucleotide with selectively hybridize to said virus nucleic acid sequence and detecting hybridization of the nucleic acid molecule in said sample, wherein the detection of the hybridization indicates the presence of virus in the sample

32. A method for detecting the presence or absence of a Chris-like virus nucleic acid molecule in a sample from a subject, comprising: (a) contacting the sample with at least one primer derived from the nucleic acid molecule according to claim 8 to provide an amplification mixture wherein said primer is sufficient in length to selectively hybridize to said virus nucleic acid sequence and wherein said primer is capable of amplifying a detectable part of a virus nucleic acid if said virus is present in the sample; (b) subjecting the amplification mixture to nucleic acid amplification and (c) detecting whether a part of a virus nucleic acid molecule has been amplified wherein detection of a part indicates the presence of virus in the sample.

33. The method according to claim 32, wherein said nucleic acid amplification is polymerase chain reaction.

34. A method for monitoring treatment or progression of a disease or disorder resulting from Chris-like virus infection in a subject, comprising (a) contacting a sample from a subject periodically with a primer or probe derived from the nucleic acid molecule of claim 8 and (b) periodically determining the amount of virus present in said subject.

35. The method according to claim 34, wherein the amount of the nucleic acid molecule present in said sample is determined by PCR or nucleic acid hybridization.

36. An agent that modulates the replication of the paramyxovirus of claim 1.

37. The agent according to claim 36, wherein said agent is selected from the group consisting of a peptide, peptide analog, polynucleotide, a ribozyme and a small interfering RNA (siRNA).

38. The agent according to claim 37, wherein said agent modulates the expression of a nucleic acid molecule hybridizing under stringent conditions to a nucleic acid molecule comprising SEQ ID NOS: 3, 7 or 8.

39. A composition comprising the nucleic acid molecule of claim 8 and a carrier or excipient.

40. A composition comprising the antibody of claim 6 and a carrier or excipient.

41. A composition comprising the polypeptide of claim 15 or immunogenic fragment thereof and a carrier or excipient.

42. A composition comprising the agent of claim 36 and a carrier or excipient.

43. A composition comprising the paramyxovirus of claim 3.

44. The composition of claim 43, wherein said paramyxovirus is an attenuated or inactivated virus.

45. A method of treating or preventing a viral infection resulting from a paramyxovirus infection comprising administering to said subject in need thereof the agent of claim 36 in an amount effective to treat said viral infection.

46. A method of inhibiting replication of a paramyxovirus comprising administering to a subject in need thereof the antibody of claim 6 in an amount effective to inhibit replication of said paramyxovirus.

47. A method of inhibiting replication of a paramyxovirus comprising administering to a subject in need thereof the polypeptide of claim 15 or immunogenic fragment thereof.

48. A method of modulating tumor growth in a subject in need thereof comprising administering to said subject the paramyxovirus of claim 1 in an amount effective to modulate said tumor growth.

49. A method for identifying an agent effective for modulating replication of paramyxovirus comprising comparing the effect of the agent of claim 36 and candidate agent on modulation of the replication of paramyxovirus.

50. A kit comprising at least one primer or probe derived from the nucleic acid molecule of claim 8

51. The kit according to claim 50, wherein said primer or probe is labeled with a detectable label.

52. The kit according to claim 50, wherein said kit comprises a plurality of primers or probes.

53. The kit according to claim 50, wherein said primers or probes are attached to a solid support.

54. The kit according to claim 50, wherein said kit further comprises enzymes.

55. A kit comprising at least one polypeptide consisting of at least 10 amino acids found in a linear array of the isolated protein or polypeptide of claim 16.

56. The kit according to claim 55 wherein said polypeptide comprises a detectable label.

57. The kit of claim 55, wherein said polypeptide is attached to a solid support.

58. The kit of claim 55, wherein further comprises one or more immunochemical reagents.

59. A nucleic acid molecule comprising a chris-like virus nucleic acid sequence operably linked to a heterologous nucleic acid sequence encoding a heterologous protein.

60. A host cell comprising the nucleic acid molecule of claim 59.

61. A method of obtaining a heterologous protein comprising culturing the host cell of claim 60 and isolating said heterologous protein.


SEQUENCE LISTING (18 sequences)

SEQ ID NO: 1 - DNA, 1690 bp, Homo sapiens
acgcggggaggaacaaggttccttcaagttgggtctattttcttctaagtttatcactgctccatagcagttataattagattaccctgatgtcagattataatcctgatgtcctccagcagcttgtcaaggatggaatcaagactatcgagcttttacagcagagtccggaagactttcagaagacgtacggcagaagtgccatccaggagccgtcgacaagagccagaatccaaagttgggagtcccgcaatcccgatcatgattacaccggtaataaaaatcagagaagcgagggagctaaagagagagcaaacaagagcgagagcgcgggaactgcttcagctgatggaggacatggagataaaccctcaaataacgggggtgactcccaaaacgagtaccaagggtcagatcaacaggtatgggatgctgcgtataatgatgggaatagtggcggagcttggggaggtcccacaggtggacttcccacggctggagaaagagggtatcctatcacaacaggaaatcaagaatttgagggataccatcccgacggtccagttgatgctcgagagtataatcagataagctccatggatcatgagatgtctgctgcagaaactttgggctcgtctactcagctaactactatgagaaacgcaacaactgatgattttgcgaaaatttttgaggaggggacacctaaagtgcaccgacggctgagggggataactgctgttgtgccggctcaaaagcaaccaggggcagtcggtggacctgttaaaaaaggcaccgacgggagtactgcatcgacacttttgggggacgtaccattatccgggagtggtgcaatccccaatgtgcacccctcactgttacaccaacccaaaaagaatgctcatgcggagaatgcccaaggcagtgtgcaagatgtatcgacgaccggggctattggccaaagtgacgaggctgcacactttgatcaggagattgaaggaaaactcaatcttgtgcttaaggaattagatctgatcagtaagaagctagactatctaccggagatcaaagaggagatcaggaatattaataagaaaataaccaatcttagcctaggactcctctacagttgaaaacttatatcaaatccatgatgatcattatccctagctcaggaaagcctgatgtgaatgacacggctgaggtgaatccagatctgaaggcagtaattgggagagataaaactagggggttgaaggaagttacaacacacaggagtgatctagaaagtttagacctcctaagcccagcaacatcagaaatagaccctaaatacctgactcaagacctggatttcactaagtctaatgccgccaattttgtcccaggcaatgatgccagctcattttataccattgtggctatgatcaagggtgaagtaactgatgtcaaacgtcagcaagagttgatcaagtgggtgtctgactccatgaacaaacaccccatgggacagatctaccgtgtcgtgagggaggctctggatgctgaaagtgacagctcctcaagctcagatagtgattagatgctaaggcaaccagcttacacattatacaattattgttgttattgtaacactggctttgaattaatctactaaattgcccaaaaaaaaaaaaaaaaaaaaaaaaaa

SEQ ID NO: 2 - protein, 347 aa, Homo sapiens [sequence omitted]

SEQ ID NO: 3 - DNA, 1690 bp, Homo sapiens; identical to SEQ ID NO: 1

SEQ ID NO: 4 - protein, 347 aa, Homo sapiens [sequence omitted]

SEQ ID NO: 5 - DNA, 2493 bp, Homo sapiens
tgagagggtggcgacattcatgattcatgtcgggaacttcctcaggaggcgaaacaaagaatactcggtcgactattgtcgtcaaaaaattgacaagatggaccttcgcttttcacttggtgcagtgggtggcctaagtcttcatgtctgtatcaaggggaagatgagcaatgcccttaaggctcagctaggatacaaaaccacgatttgttattctctcatggacaccaacccgtacctgaacaagttgatgtggaaggctgaatgcaagatcaacaaggtgactgctgttctccagccatctgtccctaaagaattcaaggtctatgaggatgtgctgattgaccacactgggaagatcatgaagtgagtgcatacaagctaggttgtttggatcctaaaaccagcatttggatatagagttctcaatttttgcactttgagctatagttttgtgcaatgttgcattgcttgtttactttaactttggcccaatataatcctccggcacgtgaattgatactcaatttcaagtcctcattcaagatgcattattaggttattctatcatttagaatagagcattctacattcaagtatatgctagaatcttggaaagatatgcaccttgttctataaaaaacttaggaggacggggtctacttcttataccattagaattagttttagagatcccttcttcgggtgcaactcaagatcaattatggaggtaactattttgacagcctacattgcagcaagtgttttcacctgttgccactcccagttggccctcagtgagctcaccaagattggcattatccctgggaggtcatatgatctcaaaacctcgactcaagcttcttatcagtacatggttataaaacttattcctaatgtgaatggcttaaacaactgtactaacagtacagtcgaatcatacaagaaaatgctaacaagactcctgaatcccattgacgtagcacttaagaagatgaaaaatgcagtctcggacaaggctccagaaaagctaggcaacgtcgagttctggggggcggtcatcggtggagttgcattaggggtggcgacttcagcgcagattacagcaggcgttgctcttcacaactccattcagaatgcaaatgctattttgacactcaaagattctatcaggcagtctaacaaagcaatacaagaattacaaacagctatgtctaccactgtcgttgtgttaaatgctctccaggaccagataaataatcagttggccccagctataaattctctaggatgccaggtggttgctaatacactaggtctaaaattaaaccagtacttcagtgaaatctcacttgtctttggcccgaatttgcgagaccccacttcagaaacactttcaatccaggcactttccagagcatttaacggtgactttgactctattgctgtctagacttaaatatgatgacagtgatttccttgattttgttggagagtgacagcatcagaggaagaataatagatgtgtctctagaagattacctaattacaattcagattgagtaccctgctctattgacaataaaggatggctactatacagacattcaatttaataagctataatactagaggaactgagtggatatctatatttcctaagcagctgttggttagagggacttacatatctaacattgatatatcacaatgtgttcttgctgcaacttctattatatgcaaatcagacactagtacccctattagttctgctacttgggcttgtgctacagggaatgtaactagctgcgccagaactagggttgtaaatgctcatgtccccaggtttgctctctcaggtggtgttatatttgctaattgtgcacctgttgtatgtaaatgccaagatccgctttatagcataaatcaagaacccaaagtgacaaatgtaatggtcgatgttgatgcctgtaaagagatgtatcttgatggattatacataacacttggtaagtcacagttaccccgtgctatgtatgccgaggatgtgtctttgggaggtccaataagcgtggatctcatagacctgggcaatgagatcaattcaatcaatagtgcaatcaataggtcggaggagcatctggatcatgcaaatgagctacttgatcaggttaatcctaagatagtcaatgtcaagaccttcggggttatgttgggattattgctgctcctgatactatggtgtgttgtgactttggtgtggctcatttgccttacaaaacaagtgagtagagtgtcttttggaggaagcatgggtagtcgggcaccgacagttaattcactctccggattcgtgggttaacataaagaaaatggtactctggttataatatacatgattagtatttgtgttgaccctttacggtacaatccagcttaggctctatcattcctatttcaataaggtgctaaccaaaaaaaaaaaaaaaaaaa

SEQ ID NO: 6 - protein, 242 aa, Homo sapiens [sequence omitted]

SEQ ID NO: 7 - DNA, 1691 bp, Paramyxovirus Chris-like Virus
acgcggggaggaacaaggttccttcaagttgggtctattttcttctaagtttatcactgctccatagcagttataattagattaccctgatgtcagattataatcctgatgtcctccagcagcttgtcaaggatggaatcaagactatcgagcttttacagcagagtccggaagactttcagaagacgtacggcagaagtgccatccaggagccgtcgacaagagccagaatccaaagttgggagtcccgcaatcccgatcatgattacaccggtaataaaaatcagagaagcgagggagctaaagagagagcaaacaagagcgagagcgcgggaactgcttcagctgatggaggacatggagataaaccctcaaataacgggggtgactcccaaaacgagtaccaagggtcagatcaacaggtatgggatgctgcgtataatgatgggaatagtggcggagcttggggaggtcccacaggtggacttcccacggctggagaaagagggtatcctatcacaacaggaaatcaagaatttgagggataccatcccgacggtccagttgatgctcgagagtataatcagataagctccatggatcatgagatgtctgctgcagaaactttgggctcgtctactcagctaactactatgagaaacgcaacaactgatgattttgcgaaaatttttgaggaggggacacctaaagtgcaccgacggctgagggggataactgctgttgtgccggctcaaaagcaaccaggggcagtcggtggacctgttaaaaaaggcaccgacgggagtactgcatcgacacttttgggggacgtaccattatccgggagtggtgcaatccccaatgtgcacccctcactgttacaccaacccaaaaagaatgctcatgcggagaatgcccaaggcagtgtgcaagatgtatcgacgaccggggctattggccaaagtgacgaggctgcacactttgatcaggagattgaaggaaaactcaatcttgtgcttaaggaattagatctgatcagtaagaagctagactatctaccggagatcaaagaggagatcaggaatattaataagaaaataaccaatcttagcctaggactcctctacagtttgaaaacttatatcaaatccatgatgatcattatccctagctcaggaaagcctgatgtgaatgacacggctgaggtgaatccagatctgaaggcagtaattgggagagataaaactagggggttgaaggaagttacaacacacaggagtgatctagaaagtttagacctcctaagcccagcaacatcagaaatagaccctaaatacctgactcaagacctggatttcactaagtctaatgccgccaattttgtcccaggcaatgatgccagctcattttataccattgtggctatgatcaagggtgaagtaactgatgtcaaacgtcagcaagagttgatcaagtgggtgtctgactccatgaacaaacaccccatgggacagatctaccgtgtcgtgagggaggctctggatgctgaaagtgacagctcctcaagctcagatagtgattagatgctaaggcaaccagcttacacattatacaattattgttgttattgtaacactggctttgaattaatctactaaattgcccaaaaaaaaaaaaaaaaaaaaaaaaaa

SEQ ID NO: 8 - DNA, 3170 bp, Paramyxovirus Chris-like Virus
gagggccaaggtacaatggccgggaatgctggaacagctgatttcctgaattcctcctgggaggaaggaggaactctcactgccattgaccctgaagcagatgagaggggaagactaattcctaagtatagagttataaaccccggtacaaacagcaggaagagtgccggttatatgtacctattggtacatggcataattgaagagaaggatgttgcaggcgcatcagcgaagaacaacaaaacatttgcagctttcccattaggcgttgggagatcaaacgctcttccccaggatcttctggatagtgtcgtagggcttgatgttaccgtgcggagaacagctgggtcaagcgagatgctagtattcggtactagtaacattagccctgtgctcacaccttggaaagacatcctgaccactggggcaatattccctgctataaaagtgtgcaacaatgttgacatggttgctgtggacaggcagcagaagtttcgctgcatcttcttgacaatcacaatgttgacagatgcgggtgtttacaaagtcccaaagaccattctcgattttcggatggctaatgcggtgtctttcaatctgcttgttgaactgctaatcggggcggacatgacagcgtccggcatcaagggcataattaatgattacggtgagagggtgacgacattcatgattcatgtcgggaacttcctcaggagacgaaacaaagaatactcggtcgactattgtcgtcaaaaaattgacaagatggaccttcgcttttcacttggtgcagtgggtggcctaagtcttcatgtctgtatcaaggggaagatgagcaatgcccttaaggctcagctaggatacaaaaccacgatttgttattctctcatggacaccaacccgtacctgaacaagttgatgtggaaggctgaatgcaagatcaacaaggtgactgctgttctccagccatctgtccctaaagaattcaaggtctatgaggatgtgctgattgaccacactgggaagatcatgaagtgagtgcatacaagctaggttgtttggatcctaaaaccagcatttggatatagagttctcaatttttgcactttgagctatagttttgtgcaatgttgcattgcttgtttactttaactttggcccaatataatcctccggcacgtgaattgatactcaatttcaagtcctcattcaagatgcattattaggttattctatcatttagaatagagcattctacattcaagtatatgctagaatcttggaaagatatgcaccttgttctataaaaaacttaggaggacggggtctacttcttataccattagaattagttttagagatcccttcttcgggtgcaactcaagatcaattatgaaggtaactattttgacagcctacattgcagcaagtgttttcacctgttgccactcccagttggccctcagtgagctcaccaagattggcattatccctgggaggtcatatgatctcaaaacctcgactcaagcttcttatcagtacatggttataaaacttattcctaatgtgaatggcttaaacaactgtactaacagtacagtcgaatcatacaagaaaatgctaacaagactcctgaatcccattgacgtagcacttaagaagatgaaaaatgcagtctcggacaaggctccagaaaagctaggcaacgtcaagttctggggggcggtcatcggtggagttgcattaggggtggcgacttcagcgcagattacagcaggcgttgctcttcacaactccattcagaatgcaaatgctattttgacactcaaagattctatcaggcagtctaacaaagcaatacaagaattacaaacagctatgtctaccactgtcgttgtgttaaatgctctccaggaccagataaataatcagttggccccagctataaattctctaggatgccaggtggttgctaatacactaggtctaaaattaaaccagtacttcagtgaaatctcacttgtctttggcccgaatttgcgagaccccacttcagaaacactttcaatccaggcactttccagagcatttaacggtgactttgactctttgctgtctagacttaaatatgatgacagtgatttccttgatttgttggagagtgacagcatcagaggaagaataatagatgtgtctctagaagattacctaattacaattcagattgagtaccctgcttctatttggacaataaaagggatggcttactataccaggacatttcaatttaataacgctataatactagaggaactgagtggatatctatatttcctaagcagctgttggttagagggacttacatatctaacattgatatatcacaatgtgttcttgctgcaacttctattatatgcaaatcagacactagtacccctattagttctgctacttgggctctgtgctacagggaatgtaactagctgcgccagaactagggttgtaaatgctcatgtccccaggtttgctctctcaggtggtgttatatttgctaattgtgcacctgttgtatgtaaatgccaagatccgctttatagcataaatcaagaacccaaagtgacaaatgtaatggtcgatgttgatgcctgtaaagagatgtatcttgatggattatacataacacttggtaagtcacagttaccccgtgctatgtatgccgaggatgtgtctttgggaggtccaataagcgtggatctcatagacctgggcaatgagatcaattcaatcaatagtgcaatcaataggtcggaggagcatctggatcatgcaaatgagctacttgatcaggttaatcctaagatagtcaatgtcaagaccttcggggttatgttgggattattgctgctcctgatactatggtgtgttgtgactttggtgtggctcatttgccttacaaaacaagtgagtagagtgtcttttggaggaagcatgggtagtcgggcaccgacagttaattcactctccggattcgtgggttaacataaagaaaatggtactctggttataatatacatgattagtatttgtgttgaccctttacggtacaatccagcttaggctctatcattcctatttcaataaggtgctaaccaaaaaaaaaaaaaaaaaaa

SEQ ID NO: 9 - protein, 340 aa, Paramyxovirus Chris-like Virus
MAGNAGTADFLNSSWEEGGTLTAIDPEADERGRLIPKYRVINPGTNSRKSAGYMYLLVHGIIEEKDVAGASAKNNKTFAAFPLGVGRSNALPQDLLDSVVGLDVTVRRTAGSSEMLVFGTSNISPVLTPWKDILTTGAIFPAIKVCNNVDMVAVDRQQKFRCIFLTITMLTDAGVYKVPKTILDFRMANAVSFNLLVELLIGADMTASGIKGIINDYGERVTTFMIHVGNFLRRRNKEYSVDYCRQKIDKMDLRFSLGAVGGLSLHVCIKGKMSNALKAQLGYKTTICYSLMDTNPYLNKLMWKAECKINKVTAVLQPSVPKEFKVYEDVLIDHTGKIMK

SEQ ID NO: 10 - protein, 497 aa, Paramyxovirus Chris-like Virus
MSDYNPDVLQQLVKDGIKTIELLQQSPEDFQKTYGRSAIQEPSTRARIQSWESRNPDHDYTGNKNQRSEGAKERANKSESAGTASADGGHGDKPSNNGGDSQNEYQGSDQQVWDAAYNDGNSGGAWGGPTGGLPTAGERGYPITTGNQEFEGYHPDGPVDAREYNQISSMDHEMSAAETLGSSTQLTTMRNATTDDFAKIFEEGTPKVHRRLRGITAVVPAQKQPGAVGGPVKKGTDGSTASTLLGDVPLSGSGAIPNVHPSLLHQPKKNAHAENAQGSVQDVSTTGAIGQSDEAAHFDQEIEGKLNLVLKELDLISKKLDYLPEIKEEIRNINKKITNLSLGLLYSLKTYIKSMMIIIPSSGKPDVNDTAEVNPDLKAVIGRDKTRGLKEVTTHRSDLESLDLLSPATSEIDPKYLTQDLDFTKSNAANFVPGNDASSFYTIVAMIKGEVTDVKRQQELIKWVSDSMNKHPMGQIYRVVREALDAESDSSSSSDSD

SEQ ID NO: 11 - protein, 546 aa, Paramyxovirus Chris-like Virus
MEVTILTAYIAASVFTCCHSQLALSELTKIGIIPGRSYDLKTSTQASYQYMVIKLIPNVNGLNNCTNSTVESYKKMLTRLLNPIDVALKKMKNAVSDKAPEKLGNVEFWGAVIGGVALGVATSAQITAGVALHNSIQNANAILTLKDSIRQSNKAIQELQTAMSTTVVVLNALQDQINNQLAPAINSLGCQVVANTLGLKLNQYFSEISLVFGPNLRDPTSETLSIQALSRAFNGDFDSIAVLDLNMMTVISLILLESDSIRGRIIDVSLEDYLITIQIEYPALLTIKDGYYTDIQFNKLLILEELSGYLYFLSSCWLEGLTYLTLIYHNVFLLQLLLYANQTLVPLLVLLLGLVLQGMLTSCARTRVVNAHVPRFALSGGVIFANCAPVVCKCQDPLYSINQEPKVTNVMVDVDACKEMYLDGLYITLGKSQLPRAMYAEDVSLGGPISVDLIDLGNEINSINSAINRSEEHLDHANELLDQVNPKIVNVKTFGVMLGLLLLLILWCVVTLVWLICLTKQVSRVSFGGSMGSRAPTVNSLSGFVG

SEQ ID NO: 12 - protein, 292 aa, Paramyxovirus Chris-like Virus
MSDYNPDVLQQLVKDGIKTIELLQQSPEDFQKTYGRSAIQEPSTRARIQSWESRNPDHDYTGNKNQRSEGAKERANKSESAGTASADGGHGDKPSNNGGDSQNEYQGSDQQVWDAAYNDGNSGGAWGGPTGGLPTAGERGYPITTGNQEFEGYHPDGPVDAREYNQISSMDHEMSAAETLGSSTQLTTMRNATTDDFAKIFEEGTPKVHRRLRGITAVVPAQKQPGAVGGPVKKGHRREYCIDTFGGRTIIREWCNPQCAPLTVTPTQKECSCGECPRQCARCIDDRGYWPK

SEQ ID NO: 13 - protein, 160 aa, Paramyxovirus Chris-like Virus
MSSSSLSRMESRLSSFYSRVRKTFRRRTAEVPSRSRRQEPESKVGSPAIPIMITPVIKIREARELKREQTRARARELLQLMEDMEINPQITGVTPKTSTKGQINRYGMLRIMMGIVAELGEVPQVDFPRLEKEGILSQQEIKNLRDTIPTVQLMLESIIR

SEQ ID NO: 14 - protein, 313 aa, Paramyxovirus Chris-like Virus
MSDYNPDVLQQLVKDGIKTIELLQQSPEDFQKTYGRSAIQEPSTRARIQSWESRNPDHDYTGNKNQRSEGAKERANKSESAGTASADGGHGDKPSNNGGDSQNEYQGSDQQVWDAAYNDGNSGGAWGGPTGGLPTAGERGYPITTGNQEFEGYHPDGPVDAREYNQISSMDHEMSAAETLGSSTQLTTMRNATTDDFAKIFEEGTPKVHRRLRGITAVVPAQKQPGAVGGPVKKGAPTGVLHRHFWGTYHYPGVVQSPMCTPHCYTNPKRMLMRRMPKAVCKMYRRPGLLAKVTRLHTLIRRLKENSILCLRN

SEQ ID NO: 15 - protein, 709 aa, Paramyxovirus Nipah Virus
MDKLELVNDGLNIIDFIQKNQKEIQKTYGRSSIQQPSIKDQTKAWEDFLQCTSGESEQVEGGMSKDDGDVERRNLEDLSSTSPTDGTIGKRVSNTRDWAEGSDDIQLDPVVTDVVYHDHGGECTGYGFTSSPERGWSDYTSGANNGNVCLVSDAKMLSYAPEIAVSKEDRETDLVHLENKLSTTGLNPTAVPFTLRNLSDPAKDSPVIAEHYYGLGVKEQNVGPQTSRNVNLDSIKLYTSDDEEADQLEFEDEFAGSSSEVIVGISPEDEEPSSVGGKPNESIGRTIEGQSIRDNLQAKDNKSTDVPGAGPKDSAVKEEPPQKRLPMLAEEFECSGSEDPIIRELLKENSLINCQQGKDAQPPYHWSIERSISPDKTEIVNGAVQTADRQRPGTPMPKSRGIPIKKGTDAKYPSAGTENVPGSKSGATRHVRGSPPYQEGKSVNAENVQLNASTAVKETDKSEVNPVDDNDSLDDKYIMPSDDFSNTFFPHDTDRLNYHADHLGDYDLETLCEESVLMGVINSIKLINLDMRLNHIEEQVKEIPKIINKLESIDRVLAKTNTALSTIEGHLVSMMIMIPGKGKGERKGKNNPELKPVIGRDILEQQSLFSFDNVKNFRDGSLTNEPYGAAVQLREDLILPELNFEETNASQFVPMADDSSRDVIKTLIRTHIKDRELRSELIGYLNKAENDEEIQEIANTVNDIIDGNI

SEQ ID NO: 16 - protein, 352 aa, Paramyxovirus Nipah Virus
MEPDIKSISSESMEGVSDFSPSSWEHGGYLDKVEPEIDENGSMIPKYKIYTPGANERKYNNYMYLICYGFVEDVERTPETGKRKKIRTIAAYPLGVGKSASHPQDLLEELCSLKVTVRRTAGSTEKIVFGSSGPLNHLVPWKKVLTSGSIFNAVKVCRNVDQIQLDKHQALRIFFLSITKLNDSGIYMIPRTMLEFRRNNAIAFNLLVYLKIDADLSKMGIQGSLDKDGFKVASFMLHLGNFVRRAGKYYSVDYCRRKIDRMKLQFSLGSIGGLSLHIKINGVISKRLFAQMGFQKNLCFSLMDINPWLNRLTWNNSCEISRVAAVLQPSIPREFMIYDDVFIDNTGRILKG

SEQ ID NO: 17 - protein, 546 aa, Paramyxovirus Nipah Virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 18 - protein, 64 aa, Paramyxovirus Nipah Virus
PGTPMPKSRGIPIKKGHRREISICWDGKRAWVEEWCNPACSRITPLPRRQECQCGECPTECFHC